SEQUENCE LISTING (3 sequences)

SEQ ID NO: 1 - DNA, 20 bp, synthetic construct
  source 1..20: mol_type = other DNA
acgtgtggctcaagtgtcaa

SEQ ID NO: 2 - DNA, 22 bp, synthetic construct
  source 1..22: mol_type = other DNA
cacttcacacctgcagtagttt

SEQ ID NO: 3 - DNA, 1849 bp, synthetic construct
  source 1..1849: mol_type = other DNA
ttgacagctagctcagtcctaggtataatgctagcattcacataaggagggatccatgcacgacctgatccagcacgctgacgctttcgttggtgacccggaccaggaatctggtggtctgtctcgtcgttctttcctgggtaaatctgctaccctgggtgctgttggtctggttgctggttggaccccggctttcgttatccagccggctgaagctgctgcttcttcttgcccggctccggctggtttcccggctggtctggaactgtaccgtcgtgctttccgtaactggtctggtgaaatcgctgctgacgacctgtggtcttgcgctccgcgtaccaacgaagaagttctggctgttgttaactgggcttggcagaacggtttcaaagttcgtccgcgtggtatgggtcacaactggtctccgctgctgctgaaaggtggtgaaaactgcgaatctcgtatcgttctggttgaaacctctcgttacctgacccgtgttcgtatcgacacccagggtgaattcggtctgttctctgctcagaccggtgttaccatggaagctctgctgaaacagctggaacgtgttaaactgggtttcgttgctaccccggctccgggtgacctgaccctgggtggtgttctggctatcgacggtcacggtaccggtatcccggctcagggtgaatctcgtctgccgggtcagtcttacggttctctgtctaactctatcgttgctctgaccgctgttgtttgggacggtgctgctggtcagtacgttctgaaaaccttccgtcgtgacgacccggcttgcgctccgttcctgacccacctgggtcgtgctttcatcgttgaagctaccctgcaggctggtgttaacaaacgtatccgttgccagtcttacgttaacatcccggcttctgaaatgttcgctgctgctggttctggtggtcgtaccttcgactctttcctgcagaaatctggtcgtgctgaagctatctggttcccgttcaccgacaaaccgtggctgaaagtttggaccccgaccccgcgttgcccgttcggtacccgtgctgttaacggtccgttcaactacccgttctctgacaacatcccgaaagctctgtctgacctgctggctgctatcaacaccggtcacccgcagctgaccccgctgctgggtaaactgcagtacgacctggttgtttctggtatggctctgaccctgggttacgacctgtggggttggtctaaagacctgctgctgtacgttaaaccgaacaccctgcgtgttaccgctaacggttacgctgttctgacccgtcgtcgtgacgttcagcgtgttatcaacgaattctacctgcagtaccagaccatggttgctgcttaccgtgctaacggtcactacccgatgaacggtccggttgaaatccgtgtttctggtctggaccagccgggtgaatctatcgttccgggtgctcaggttccgtctctgtctgctatccgtccgcgtccggaccagccggaatgggacaccgctatctggctggacatcctgtctctgccgggtaccccgcaggctaacgctttctaccacgaattcgaagcttggctgttcgaccacttctctggtgactacgcttctctgcgtgttgaatggtctaaaggttggggttactctccggctgctgcttgggacgaaccgaccgttgttgaccagctggttgctcagtctctgcgtcagggtctggttgctgacaacgactgggactctgctgttcgtcagctgaacgaagctgacccgcaccgtctgttctcttctccgctgctggaccgtctgatgccgtag

(19) United States
(12) Patent Application Publication
(10) Pub. No.: US 2026/0077000 A1
(43) Pub. Date: Mar. 19, 2026

(54) METABOLIC MODULATION OF INTRATUMORAL CHOLESTEROL WITH ENGINEERED BACTERIA FOR THE TREATMENT OF CANCERS

(71) Applicant: Hong Kong Baptist University, Hong Kong (CN)

(72) Inventors: Hoi Leong Xavier WONG, Hong Kong (CN); Jiayan WU, Hong Kong (CN); Lixiang ZHAI, Hong Kong (CN); Pallavi ASTHANA, Hong Kong (CN)

(21) Appl. No.: 19/330,914

(22) Filed: Sep. 17, 2025

Related U.S. Application Data
(60) Provisional application No. 63/695,383, filed on Sep. 17, 2024.

(51) Int. Cl. A61K 35/74; A61K 31/575; A61K 45/06; A61P 35/00; C07K 16/28; C12N 9/04; C12N 15/70; C12R 1/19

(52) U.S. Cl. CPC A61K 35/74; A61K 31/575; A61K 45/06; A61P 35/00; C07K 16/2863; C12N 9/0006; C12N 15/70; C12R 2001/19; C12Y 101/03006

(57) ABSTRACT

A method of treating a cancer (e.g., a colorectal cancer, a liver cancer, a breast cancer, or a lung cancer) in a subject in need thereof by administering to the subject a therapeutically effective amount of (i) a bacterium that converts intestinal cholesterol to 4-cholesten-3-one (4-C-3) or (ii) 4-C-3. Also disclosed is a recombinant Escherichia coli TOP10 bacterium comprising a nucleic acid encoding cholesterol oxidase (CHOX), which is capable of converting cholesterol to 4-C-3 and can be used to treat gastrointestinal tract cancers including a colorectal cancer or lung cancer.

### CROSS-REFERENCE TO RELATED APPLICATIONS

The present application claims priority from U.S. Provisional Patent Application No. 63/695,383, filed on Sep. 17, 2024, which is hereby incorporated by reference in its entirety.

### TECHNICAL FIELD

The present disclosure relates to the use of (i) a bacterium that converts intestinal cholesterol into 4-cholesten-3-one (4-C-3) or (ii) 4-C-3 for the treatment of cancers, including colorectal cancer, liver cancer, breast cancer, or lung cancer.

### REFERENCE TO SEQUENCE DISCLOSURE

The sequence listing file under the file name “SEQUENCE_LISTING.xml” submitted in ST.26 XML file format with a file size of 5,578 bytes created on Dec. 4, 2025 is incorporated herein by reference.

### BACKGROUND

Colorectal cancer (CRC) ranks as the third most common cancer globally, accounting for approximately 1.85 million new cases and 850,000 deaths annually. This prevalence emphasizes that CRC represents a significant challenge for healthcare systems worldwide. The treatment of CRC has evolved significantly over the years, but there are still numerous limitations and challenges that affect patient outcomes. CRCs can develop resistance to chemotherapy and other drug treatments, making them less effective and limiting options for patients who have recurrent or metastatic disease. In addition, chemotherapy, radiation therapy, and surgery can have significant side effects that can impact the quality of life of patients with CRC.

Cholesterol is an essential component of cell membranes and a precursor for the synthesis of steroid hormones and bile acids. Despite its importance in physiological functions, altered cholesterol metabolism has been associated with the development of cancers, including CRC. Cholesterol accumulation is a general feature of cancer tissues, including CRC tissues. Therefore, cholesterol-lowering strategies have therapeutic potential for cancer prevention and treatment. Safe therapeutic approaches for modulating cholesterol levels thus are important in the treatment of cancers, including CRC.

It has become increasingly clear that microbes residing in the gastrointestinal tract, also known as the gut microbiota, can play a significant role in the development and progression of CRC. Certain pathogenic strains of bacteria, such as Fusobacterium nucleatum and Bacteroides fragilis, are associated with an increased risk of CRC. The maintenance of a balanced microbial community is therefore crucial for preventing the overgrowth of protumorigenic bacterial species, reducing the risk of inflammation-related carcinogenesis.

Recent studies suggest that certain species of human gut bacteria, such as Oscillibacter spp. and Eubacterium coprostanoligenes, can break down cholesterol in the gut, contributing to the reduced risk of cardiovascular diseases in people. Some bacterial species can also affect the efficiency of cholesterol absorption from the diet. While the gut microbiota can affect cholesterol levels and potentially influence the risk of conditions such as CRC, the relationship is complex and multifactorial. Gut microbiota can promote the conversion of intestinal cholesterol into coprostanol for excretion. The idea that gut bacteria can control cholesterol levels has been proposed over a century. However, the exact role of this process is still unknown. Further research is needed to fully understand the mechanisms by which the gut microbiota influences CRC and to elucidate the intricate connections among the gut microbiota, cholesterol metabolism, and cancers (including CRC), in order to explore an approach for treating cancers characterized in high levels of cholesterol, including CRC.

### SUMMARY

In a first aspect, the present disclosure provides a method of treating a cancer in a subject in need thereof, the method comprising administering to the subject a therapeutically effective amount of (i) a bacterium that converts intestinal cholesterol to 4-cholesten-3-one (4-C-3) or (ii) 4-C-3.

In certain embodiments, the bacterium is selected from the group consisting of Oscillibacter species expressing cholesterol oxidase (CHOX), Eubacterium coprostanoligenes, a recombinant bacterium comprising a nucleic acid encoding CHOX, and any combination thereof, and wherein the recombinant bacterium is selected from the group consisting of bacteria of the genus Escherichia and genus Bacillus.

In certain embodiments, the Oscillibacter species is Oscillibacter ruminantium and the recombinant bacterium is selected from the group consisting of an Escherichia coli bacterium, a Bacillus subtilis bacterium, and any combination thereof.

In certain embodiments, the recombinant bacterium is selected from the group consisting of E. coli TOP10, E. coli DH5a, E. coli BL21, B. subtilis 168, and any combination thereof.

In certain embodiments, the CHOX is selected from the group consisting of CHOX originating from Streptomyces species, such as Streptomyces fradiae, Streptomyces lavendulae, Streptomyces cavourensis, or any combination thereof.

In certain embodiments, the 4-C-3 or the bacterium is administered in a therapeutically effective amount to achieve one or more or all of the following:
(i) reduce cholesterol concentrations within tumor tissues, gastrointestinal tract, serum, or feces of the subject;
(ii) increase the 4-C-3 concentrations within tumor tissues, gastrointestinal tract, serum, or feces of the subject;
(iii) inhibit the growth or viability of cancer cells;
(iv) reduce the size, weight, or volume of cancer tumors;
(v) promote apoptosis of cancer cells;
(vi) antagonize the EGFR pathway; and
(vii) target KRAS mutants.

In certain embodiments, a fecal sample, a tumor sample, a plasm sample, or a serum sample obtained from the subject prior to administering a therapeutically effective amount of the bacterium or 4-C-3 exhibits at least one of: an increased cholesterol concentration and a decreased 4-C-3 concentration.

In certain embodiments, the method further comprises providing a sample obtained from the subject, wherein the sample is a fecal sample, a tumor sample, a plasm sample, or a serum sample; determining the concentration of at least one of 4-C-3 and cholesterol in the sample; and administering the 4-C-3 or the bacterium to the subject if a decreased concentration of 4-C-3 or an increased concentration of cholesterol is detected in the sample relative to an average concentration detected in healthy subjects.

In certain embodiments, the method further comprises comprising co-administering one or more additional agents selected from the group consisting of: 5-cholesten-3-one, coprostanone, statins, chemotherapeutic agents, radiation therapeutic agents, immunotherapeutic agents, anti-EGFR therapeutic agents, agents targeting KRAS mutations, and any combination thereof.

In certain embodiments, the method further comprises co-administering anti-EGFR therapeutic agents, wherein the anti-EGFR therapeutic agent is selected from the group consisting of cetuximab, panitumumab, necitumumab, erlotinib, gefitinib, osimertinib, afatinib, dacomitinib, lapatinib, and any combination thereof.

In certain embodiments, the cancer is selected from the group consisting of a gastrointestinal cancer, a lung cancer, a breast cancer, and any combination thereof.

In certain embodiments, the gastrointestinal cancer comprises a gastrointestinal tract cancer selected from the group consisting of esophageal cancer, esophageal adenocarcinoma, gastric cancer, gastric adenocarcinoma, small intestine cancer, gastrointestinal lymphoma, colon cancer, rectal cancer, colorectal cancer, colorectal adenocarcinoma, anal cancer, appendiceal cancer, pancreatic cancer, pancreatic adenocarcinoma, and any combination thereof; a biliary tract cancer selected from the group consisting of a gallbladder cancer, a cholangiocarcinoma, and any combination thereof; a liver cancer; or any combination thereof.

In certain embodiments, the lung cancer is selected from the group consisting of non-small cell lung cancer such as lung adenocarcinoma, small cell lung cancer, and any combination thereof.

In certain embodiments, the breast cancer is selected from the group consisting of hormone receptor-positive breast cancer, triple-negative breast cancer, HER2-enriched breast cancer, and any combination thereof.

In certain embodiments, the gastrointestinal tract cancer or lung cancer is characterized by the presence of a KRAS mutation. In certain embodiments, the KRAS mutation is selected from the group consisting of G12A, G12C, G12D, G12E, G12F, G12I, G12R, G12S, G12V, G13A, G13C, G13D, G13E, G13R, and any combination thereof.

In certain embodiments, the cancer is a colorectal cancer, a lung cancer, a liver cancer, or a breast cancer, and the bacterium is Oscillibacter ruminantium or a recombinant Escherichia coli bacterium comprising a nucleic acid encoding cholesterol oxidase (CHOX).

In certain embodiments, the 4-C-3 or bacterium is administered orally.

In certain embodiments, the bacterium is administered via fecal microbiota transplant.

In certain embodiments, the subject is a human, a non-human primate, a rodent, a canine, a feline, a bovine, or an equine.

In a second aspect, provided herein is a recombinant Escherichia coli TOP10 bacterium comprising a nucleic acid encoding cholesterol oxidase (CHOX), optionally, wherein the nucleic acid is operably linked to a promoter such that the bacterium expresses the CHOX under the control of the promoter.

In certain embodiments, the promoter is a constitutive promoter J23119, and wherein the CHOX is CHOX originating from Streptomyces species such as Streptomyces fradiae, Streptomyces lavendulae, or Streptomyces cavourensis.

In a third aspect, provided herein is a composition comprising the bacterium of the second aspect and a pharmaceutically acceptable carrier.

In certain embodiments, the composition is in the form of a lyophilized powder, a freeze-dried preparation, a microencapsulation, an enteric-coated capsule, a tablet, a granule, a suspension, or any combination thereof.

### BRIEF DESCRIPTION OF DRAWINGS

The above and other objects and features of the present disclosure will become apparent from the following description of the disclosure, when taken in conjunction with the accompanying drawings.

FIG. 1 shows the level of cholesterol and its microbial metabolites in fecal samples of germ-free mice and healthy individuals. (a-e) The relative level of cholesterol and its indicated microbial metabolites in germ-free mice and conventional mice. Data (n=10 in each group) are expressed as the mean±SEM. The significant differences were analyzed by unpaired t-tests. Legend in (a-e), hollow square (E): conventional mice; hollow circle (O): germ-free mice. (f) Physiological levels of cholesterol and its indicated microbial metabolites in healthy human individuals. Data (n=7 in each group) are expressed as the mean±SEM.

FIG. 2 shows reduced 4-C-3 in human colon cancer and anti-tumor activity of 4-C-3 in human colorectal cancer cells. (a-e) Levels of cholesterol and its microbial metabolites 5-cholesten-3-one (5-C-3), 4-cholesten-3-one (4-C-3), coprostanone, and coprostanol in CRC tumor biopsies and in adjacent normal tissues. Legend in (a-e), hollow square (□): CRC tumor biopsies; hollow circle (∘): normal adjacent tissues. Data (n=12 in each group) are expressed as the mean±SEM. The significant differences were analyzed by unpaired t-tests. (f-g) Fecal cholesterol and 4-C-3 levels in a CRC patient cohort with 532 samples. Data are expressed as the mean±SEM. The significant differences were analyzed by unpaired t-tests. (h-n) MTT assay in a panel of human colorectal cancer cell lines (HCT15, HT55, colo205, HCT116, DLD-1, Caco2 and YAMC) treated with indicated microbial metabolites of cholesterol at indicated dosages for 48 h. Data (n=3 in each group) are expressed as the mean±SEM. Legend in (h-n), solid square (▪) 5-C-3; solid circle (●): 4-C-3; solid upright triangle (▴): Coprostanone; solid inverted triangle (▾): Coprastanol. (o-p) Flow cytometry analyses of cell apoptosis in DLD-1 and HCT116 cells treated with vehicle (0.1% ethanol) or indicated microbial metabolites of cholesterol for 48 h. The quantification of apoptotic rates is shown in (p). Data (n=3) are expressed as the mean±SEM and the significant differences were analyzed by one-way ANOVA. Legend in (p), hollow inverted triangle (∇): vehicle; hollow square (□): 5-C-3; hollow circle (∘): 4-C-3; hollow upright triangle (Δ): Coprastanone. (q) Cell viability of colorectal cancer organoids derived from two patients with CRC upon the treatment with 4-C-3 at indicated dosages or 0.1% ethanol as control. Data (n=3) are expressed as the mean±SEM and the significant differences were analyzed by one-way ANOVA.

FIG. 3 shows 4-C-3 suppresses colon tumorigenesis in vivo by 4-C-3 or vehicle (10% ethanol+90% corn oil). (a) Schematic diagram showing the experimental design for establishing subcutaneous CRC model with DLD-1 cells and the treatment with 4-C-3 at indicated dosages. (b) Images of DLD-1 xenograft tumors after the treatment with 4-C-3 for 16 days for different groups M1, M2, M3, M4, and M5. The scale bar shown below the panel represents measurements in centimeters (cm). (c) Tumor volume and (d) tumor weight of DLD-1 xenograft tumors of mice in the indicated groups. Data (n=5) are expressed as the mean±SEM and the significant differences were analyzed by two-way ANOVA for (c) and one-way ANOVA for (d). (e) Schematic diagram showing the experimental design for establishing subcutaneous CRC model with MC38 cells and the treatment with 4-C-3 at indicated dosages. (f) Images of MC38 xenograft tumors after the treatment with 4-C-3. Tumor weight (g) and tumor volume (h) of MC38 xenograft tumors of mice in the indicated groups. Data (n=6) are expressed as the mean±SEM and the significant differences were analyzed by two-way ANOVA for (g) and one-way ANOVA for (h). (i) Schematic representation showing the experimental design for establishing orthotopic CRC model with luciferase-labelled HCT116 cells and the treatment with 4-C-3 at indicated dosages. (j) Images for luciferase signal of HCT116 xenograft tumors after the treatment with 4-C-3. (k) Quantification of luciferase signal emitted by HCT116 xenograft tumors for the measurement of tumor growth. (1) Tumor weight. Data (n=3) are expressed as the mean±SEM and the significant differences were analyzed by two-way ANOVA for (k) and one-way ANOVA for (l). Legend in (c, g, k), solid inverted triangle (▾): Vehicle; solid square (▪) 4-C-3 at 10 mg kg⁻¹; solid upright triangle (▴): 4-C-3 at 25 mg kg⁻¹; and solid circle (●): 4-C-3 at 50 mg kg⁻¹. Legend in (d, h, 1), hollow inverted triangle (∇): vehicle; hollow square (□): 4-C-3 at 10 mg/kg; hollow upright triangle (Δ): 4-C-3 at 25 mg/kg; and hollow circle (∘): 4-C-3 at 50 mg/kg. 10% ethanol+90% corn oil was used as vehicle.

FIG. 4 shows the antitumor effect of 4-cholesten-3-one (4-C-3) on orthotopic xenografts of CRC cells. (a-e) The level of intratumoral 4-C-3 (a), the proliferation of cancer cells assessed by immunostaining of Ki-67 (b-c) and the apoptosis of cancer cells by TUNEL assay (d-e) in orthotopic xenografts of HCT116 cells upon 4-C-3 treatment for 25 days. Data (n=3) are expressed as the mean±SEM and the significant differences were analyzed by one-way ANOVA for a, c & e. Legend in (a, c, e), hollow inverted triangle (∇): vehicle; hollow square (□): 4-C-3 at 10 mg/kg; hollow upright triangle (Δ): 4-C-3 at 25 mg/kg; and hollow circle (∘): 4-C-3 at 50 mg/kg. 10% ethanol+90% corn oil was used as vehicle.

FIG. 5 shows the safety profile of 4-cholesten-3-one (4-C-3) in mice. (a-c) The changes in body weight in mice bearing DLD-1 xenograft tumors (a), orthotopic HCT116 xenografts (b), and patient derived xenograft (PDX) tumors (c) with the treatment of 4-C-3 at indicated dosages. Data n=5 for (a), n=3 for (b), and n=6 for (c) are expressed as the mean±SEM and the significant differences were analyzed by two-way ANOVA. (d-f) C57BL mice at the age of 8 weeks were treated with 4-C-3 (50 mg/kg) for 4 weeks. The changes in body weight in mice were shown in (d). (e-f) The blood samples were collected for regular blood tests and different parameters were shown in (e-f). Legend in (a, b), solid inverted triangle (▾): vehicle; solid square (▪): 4-C-3 at 10 mg/kg; solid upright triangle (▴): 4-C-3 at 25 mg/kg; and solid circle (●): 4-C-3 at 50 mg/kg.). Legend in (c, d), solid inverted triangle (▾): Vehicle; and solid circle (●): 4-C-3 at 50 mg kg⁻¹. 10% ethanol+90% corn oil was used as vehicle.

FIG. 6 shows 4-C-3 suppresses colon tumorigenesis through suppression of EGFR signaling. (a-c) RNA sequencing analyses of DLD-1 cells treated with 4-C-3 (50 μM) for 48 h. Results of (a) Volcano plot analysis, (b) KEGG pathway enrichment analysis and (c) gene set enrichment analysis. In (a), dots located on the left side of the first dashed line indicate those down-regulated (326); dots between the two dashed lines indicate those not significantly changed; and dots located on the right side of the second dashed lines indicate those up-regulated (129). (d) qPCR analysis of the expression of EREG in DLD-1 cells treated with control (0.1% ethanol) or 4-C-3 at indicated dosages for 48 h. Data (n=3) are expressed as the mean±SEM and the significant differences were analyzed by one-way ANOVA. (e-f) Western blotting analyses of EGFR signaling pathway in (e) in vitro DLD-1 cells treated with 4-C-3 at indicated dosages (n=3) and (f) DLD-1 subcutaneous xenografts upon the treatment with control (10% ethanol+90% corn oil) or 4-C-3 at indicated dosages (n=3 for each treatment group). (g) MTT assay of Caco-2 cells with siRNA-mediated knockdown of EGFR in response to the treatment with control (0.1% ethanol) or 4-C-3 at the indicated dosage. Data (n=3) are expressed as the mean±SEM and the significant differences were analyzed by one-way ANOVA. (h) Representative images for western blotting analyses for cellular thermal shift assay of EGFR in HCT116 and DLD-1 cells upon the treatment with control (0.1% ethanol) or 4-C-3 (50 μM). (n=3). (i-k) Microscale thermophoresis (MST) analyses for (i) the binding between the recombinant extracellular domain of EGFR (EGFR-ECD) and 4-C-3; (j) the binding between EGFR-ECD and recombinant EREG and (k) the binding between EGFR-ECD and recombinant EREG in the presence of 4-C-3. (n=3). (l) Co-immunoprecipitation of EREG and EGFR in lysates of DLD-1 cells treated with 4-C-3, followed by western blotting using antibodies indicated. (n=3).

FIG. 7 shows the blockage of ligand binding of EGFR by 4-C-3. (a-b) Microscale thermophoresis (MST) analyses for (a) the binding between the recombinant extracellular domain of EGFR (EGFR-ECD) and recombinant EGF and (b) the binding between EGFR-ECD and recombinant AREG in the presence/absence of 4-C-3. (n−3).

FIG. 8 shows the structural and interactional features of EGFR in the presence of 4-C-3. (a) Molecular docking analysis of the binding between EGFR and 4-C-3. (b-e) 100 ns molecular dynamics (MD) simulations coupled with mechanics and thermodynamic calculations to study the dynamical structural characteristics and interactions between EGFR and EREG in the presence of 4-C-3. In (b), top line: Van der Waals (vdW); bottom line: Coulomb.

FIG. 9 shows 4-C-3 overcomes primary resistance to anti-EGFR therapeutics in CRC. (a) MTT assay of Caco-2 cells expressing wild-type EGFR and indicated EGFR mutants treated with either 4-C-3 and cetuximab. Data (n=3) are expressed as the mean±SEM. (b-c) Molecular docking analyses of the binding between EGFR^{WT}/EGFR^{S492R} and cetuximab (b) and the binding between EGFR^{S492R} and 4-C-3 (c). (d) MTT assay of HCT116 and LS513 cells in response to the treatment with 4-C-3 or cetuximab. Data (n=3) are expressed as the mean±SEM. (e) Western blotting analyses of EGFR and its downstream signaling pathways in Caco-2, LSS13, and HCT116 cells upon the treatment with control (0.1% ethanol) or 4-C-3 or cetuximab (n=3). (f-k) Nude mice bearing subcutaneous LS513 or HCT116 xenografts were treated with control (10% ethanol+90% corn oil), cetuximab or 4-C-3. (f, i) Images of tumors for indicated groups are shown. (g) Tumor volume and (h) tumor weight of LS513 xenografts. (j) Tumor volume and (k) tumor weight of HCT116 xenografts. Data (n=5 for each treatment group) are expressed as the mean±SEM and the significant differences were analyzed by two-way ANOVA for (g & j) and one-way ANOVA for (h & k). (l) Cell viability of CRC organoids derived from Apc^{min/+} Kras^{G12D/+} mutant mice upon the treatment with vehicle (10% ethanol+90% corn oil)) or 4-C-3 and KRAS^{G12D} inhibitor MRTX1133. Data (n=3) are expressed as the mean±SEM and the significant differences were analyzed by one-way ANOVA (m) Western blotting analyses of EGFR and its downstream signaling pathways in CRC organoids upon the treatment with vehicle (0.1% ethanol) or 4-C-3 or MRTX1133 (n=3). Legend in (a, d, g-h, j-k), solid square (▪): vehicle (0.1% ethanol); solid circle (●): Cetuximab; solid upright triangle (▴): 4-C-3; solid inverted triangle (▾): 4-C-3+ Cetuximab. Legend in (1), solid square (▪) vehicle (0.1% ethanol); solid upright triangle (▴): 4-C-3; solid circle (●): MRTX1133.

FIG. 10 shows 4-C-3 is a non-covalent inhibitor for oncogenic KRAS mutants. (a) Microscale thermophoresis (MST) analyses for the binding between recombinant KRAS^{WT}/KRAS^{G12D} bound with GDP and 4-C-3. (n=3). (b-c) The effect of 4-C-3 treatment on (b) the intrinsic and (c) SOS1-mediated nucleotide exchange in KRAS^{WT}/KRAS^{G12D}. (n=3). (d) KRAS^{WT}/KRAS^{G12D} were pre-loaded with the non-hydrolyzable GTP analogue GMPPNP (GNP), followed by the reaction with a GST-tagged RBD domain of CRAF. The mixtures were then analyzed by GST pull-down and immunoblotting for quantification with densitometry. (n=3). (e) Representative images for western blotting analyses for cellular thermal shift assay of KRAS in LS513 and Caco-2 cells upon the treatment with control (0.1% ethanol) or 4-C-3 (50 μM). (n=3). (f) LS513 cells, a KRAS G12D mutant cell line, were treated with indicated treatments and their extracts were subjected to RBD pull-down and immunoblotting to determine the level of active KRAS (KRAS-GTP). (n=3). (g) Molecular docking analysis of the binding between KRAS^{WT}/KRAS^{G12D} and 4-C-3. (h). Docking analysis of 4-C-3 with different KRAS mutants. (i) The fluorescent GDP→GTP exchange assays show 4-C-3 inhibits activation of multiple KRAS mutants (G12C, G13C, G13D) with potency comparable to G12D, indicating broad mutant coverage. Legend in (i), solid circle (●): KRAS^{G12C}; solid square (▪) KRAS^{G13C}; solid upright triangle (▴): KRAS^{G13D}.

FIG. 11 shows computational stimulation for the binding of 4-C-3 to oncogenic KRAS variants. (a-d) Long timescale simulations were performed to reveal the interaction dynamics of the 4-C-3-KRAS^{G12D} complex. The analysis results of the protein backbone root-mean-square fluctuation (RMSF) for (a-b) the 4-C-3-KRAS^{G12D} complex and (c-d) the 4-C-3-KRAS^{WT} complex. (e-f) Microscale thermophoresis (MST) analysis results for the binding of GDP on (e) KRAS^{G12D} and (f) KRAS^{WT} in the presence/absence of 4-C-3 FIG. 12 shows construction of engineered microbes for the conversion of cholesterol into 4-C-3. (a) The construction map for the construct for expressing Chox1. (b-c) The in vitro conversion of cholesterol into 4-C-3 by indicated engineered bacteria including E. coli. (TOP10), E. coli. (JM109) and E. coli. (Nissle 1917), as assessed by LC-MS analyses. PBS was used as control. (d) The optimized gene sequence of CHOX (SEQ ID NO: 3). Data (n=3 for each treatment group) are expressed as the mean±SEM and the significant differences were analyzed by one-way ANOVA. Legend in (b) hollow square (□): cholesterol; hollow circle (∘) 4-C-3.

FIG. 13 shows anti-tumour activity of engineered E. coli CHOX⁺ bacteria in producing 4-C-3. (a-d) MC38 colon cancer cells were subcutaneously implanted into nude mice to generate xenograft tumors. The mice were then gavaged daily with E. coli CHOX⁺ bacteria or E. coli CHOX-bacteria for 13 days. Images of MC38 xenografts for indicated treatment groups are shown in (a). (b) Tumor volume, (c) tumor weight and (d) the level of intratumoral 4-C-3 of MC38 xenografts for indicated treatment groups. Data (n=5 for each treatment group) are expressed as the mean±SEM and the significant differences were analyzed by two-way ANOVA for (b) and unpaired t-test for (c) and (d). Legend in (b-d), solid inverted triangle (▾): E. coli CHOX⁻ treatment; solid square (▪) E. coli CHOX⁺ treatment.

FIG. 14 shows the safety profile of E. coli CHOX⁺ bacteria in mice. (a) The changes in body weight of MC38 implanted mice upon the treatment E. coli CHOX⁺ bacteria or E. coli CHOX-bacteria for 13 days. Data (n=5) are expressed as the mean±SEM and the significant differences were analyzed by two-way ANOVA. Legend in (a), hollow inverted triangle (∇): E. coli CHOX⁻ bacteria; hollow square (□): E. coli CHOX′ bacteria. (b-d) C57BL/6 mice at the age of 8 weeks were daily gavaged with E. coli CHOX⁺ or E. coli CHOX⁻ bacteria (5×10⁸) for 4 weeks. The changes in body weight in mice were shown in (b). The blood samples were collected for regular blood tests for (c) haematological and (d) biochemical analyses. Data (n=4 for each treatment group) are expressed as the mean±SD. Legend in (b), solid inverted triangle (▾): E. coli CHOX-bacteria; and solid circle (●): E. coli CHOX⁺ bacteria.

FIG. 15 shows the efficacy of E. coli CHOX⁺ bacteria for converting cholesterol into 4-C-3. The in vitro conversion of cholesterol into 4-C-3 by E. coli CHOX⁻, E. coli CHOX⁺, O. ruminantium and Oscillibacter valericigenes bacteria were assessed by LC-MS analyses. Data (n=3 for each treatment group) are expressed as the mean±SEM and the significant differences were analyzed by one-way ANOVA.

FIG. 16 shows suppression of colon tumorigenesis by engineered microbe-mediated conversion of cholesterol to 4-C-3. (a) The levels of O. ruminantium in patients with CRC and healthy individuals. Data (n=532) are expressed as the mean±SEM and the significant differences were analyzed by unpaired t-tests. (b-f) Orthotopic CRC model with luciferase-labelled HCT116 cells were subcutaneously implanted into nude mice to generate xenograft tumors. The mice were then treated daily with O. ruminantium, O. valericigenes, E. coli CHOX⁺ bacteria or 4-C-3 for 21 days. 10% ethanol+90% corn oil was used as the vehicle. (b) Images for luciferase signal of HCT116 xenograft tumors after the treatment. (c) Quantification of luciferase signal emitted by HCT116 xenograft tumors for the measurement of tumor growth. (d) Tumor weight. (e) The level of intratumoral cholesterol and (f) the level of intratumoral 4-C-3. Data (n=5 for each treatment group) are expressed as the mean±SEM and the significant differences were analyzed by two-way ANOVA for (c) and one-way ANOVA for (d-f). Legend in (c-f), solid inverted triangle (▾): vehicle; solid square (▪) O. ruminantium; solid rhombus (♦): O. valericigenes; solid upright triangle (▴): E. coli CHOX⁺ bacteria; and solid circle (·): 4-C-3. (g-k) C57BL/6 mice were treated with antibiotics and AOM-DSS regimen, followed by daily gavage with indicated treatment groups (g). (h) The colon tumor number, (i) colon length, (j) the level of intratumoral cholesterol, and (k) the level of intratumoral 4-C-3 in the colon tissue of indicated treatment groups. Data (n=8 for each treatment group) are expressed as the mean±SEM and the significant differences were analyzed by one-way ANOVA for (h-k). Legend in (h-k), crossed circle (⊗): vehicle; hollow-solid square ( [symbol] ): AOM/DSS; hollow square (□): AOM/DSS+O. ruminantium; hollow rhombus (⋄) AOM/DSS+O. valericigenes; hollow hexagon ( [symbol] ): AOM/DSS+CHOX⁻; hollow upright triangle (Δ): AOM/DSS+CHOX⁺; and hollow circle (∘): AOM/DSS+4-C-3. 10% ethanol+90% corn oil was used as vehicle. (l-p) Immunocompetent mice were subcutaneously implanted with CRC organoids from Apc^{min/+}Kras^{G12D/+} mutant mice and were intratumorally injected with O. ruminantium, O. valericigenes, E. coli CHOX⁻ bacteria, E. coli CHOX⁺ bacteria, 4-C-3 or the KRAS^{G12D} inhibitor MRTX1133 for 10 days. (l) Images of tumors for indicated treatment groups are shown. (m-p) Tumor volume (m) and tumor weight (n), the level of intratumoral cholesterol (o), and the level of intratumoral 4-C-3 (p). Data (n=5 for each treatment group) are expressed as the mean±SEM and the significant differences were analyzed by two-way ANOVA for (m) and one-way ANOVA for (n-p). Legend in (m-p), crossed circle (⊗): vehicle; hollow square (▪) O. ruminantium; hollow rhombus (⋄): O. valericigenes; hollow hexagon ( [symbol] ): CHOX⁻; hollow upright triangle (Δ): CHOX⁺; hollow circle (∘): 4-C-3; and hollow-solid square ( [symbol] ): MRTX1133. 10% ethanol+90% corn oil was used as vehicle.

FIG. 17 shows anti-tumor activity of 4-C-3 in human cancer cells. MTT assay was conducted in a panel of human colorectal cancer cell lines (A549, Calu-3, H1299, LA795, and LLC), human breast cancer cell lines (HCC1806, MCF-7, MDA-MB-231), and human liver cancer cell lines (Hepg2, Hep3b, HUH7) treated with 4-C-3 at indicated dosages for 48 h. Legend, solid square (▪): vehicle (0.1% ethanol) treated mice; solid triangle (▴): 4-C-3 treated mice. Data (n=3) are expressed as the mean±SEM.

FIG. 18 shows anti-tumour activity of engineered E. coli CHOX⁺ bacteria in breast and lung cancer mouse models. C57BL/6 male mice were subcutaneously inoculated with E0771 breast cancer cells (a-d) or LLC lung cancer cells into both flanks (e-h). The mice were subsequently administered daily intratumoral injections of E. coli CHOX⁺ bacteria, E. coli CHOX-bacteria (2×10⁷ CFU/flank) or 4-C-3. 10% ethanol+90% corn oil was used as the vehicle. (a-d) Images of EO771 xenografts for indicated treatment groups, (b) Tumor weight, (c) tumor volume and (d) the level of intratumoral 4-C-3 of E0771 xenografts for indicated treatment groups. (e-h) Images of LLC xenografts for indicated treatment groups, (f) Tumor weight, (g) tumor volume and (h) the level of intratumoral 4-C-3 of LLC xenografts for indicated treatment groups. Data (n=6 for each treatment group) are expressed as the mean±SEM and the significant differences were analyzed by two-way ANOVA for (c) and (g) and one-way ANOVA for (b, d, f and h) and (d). Legend, solid square (▪) Vehicle; solid circle (●): 4-C-3; solid upright triangle (▴): E. coli CHOX-bacteria; and solid inverted triangle (▾): E. coli CHOX⁺ bacteria.

### DETAILED DESCRIPTION

### Definitions

Throughout the present specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present specification and claims, unless the context requires otherwise, the word “include” or variations such as “includes” or “including”, will be understood to imply the inclusion of a stated integer or group of integers but not the exclusion of any other integer or group of integers.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “subject” as used herein, refers to an animal, typically a mammal or a human, that will be or has been the object of treatment, observation, and/or experiment. When the term is used in conjunction with administration of a compound described herein, then the subject has been the object of treatment, observation, and/or administration of the compound described herein.

The term “therapeutically effective amount” as used herein, means that amount of the compound or pharmaceutical agent that elicits a biological and/or medicinal response in a cell culture, tissue system, subject, animal, or human that is being sought by a researcher, veterinarian, clinician, or physician, which includes alleviation of the symptoms of the disease, condition, or disorder being treated.

As used herein, the term “isolated” in connection with a compound described herein means the compound is separated from other components of either a natural source, such as a plant or cell; or a synthetic organic reaction mixture, such as by conventional techniques.

As used herein, the term “substantially pure” in connection with a sample of a compound described herein means the sample contains at least 60% by weight of the compound. In certain embodiments, the sample contains at least 70% by weight of the compound; at least 75% by weight of the compound; at least 80% by weight of the compound; at least 85% by weight of the compound; at least 90% by weight of the compound; at least 95% by weight of the compound; or at least 98% by weight of the compound.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±9%, ±8%, ±7%, ±6%, ±5%, ±4%, ±3%, ±2%, ±1%, or ±0% variation from the nominal value unless otherwise indicated or inferred.

Provided herein is a method of treating a cancer (such as a gastrointestinal tract cancer, a lung cancer, a liver cancer, or a breast cancer) in a subject in need thereof, the method comprising administering to the subject a therapeutically effective amount of (i) a bacterium that converts intestinal cholesterol to 4-cholesten-3-one (4-C-3) or (ii) 4-C-3.

The compound 4-C-3 can have a structure of

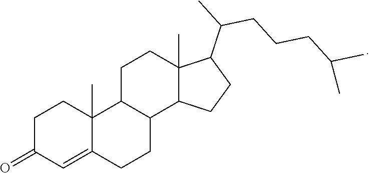 [SMILES: CC(C)CCCC(C)C1CCC2C3CCC4=CC(=O)CCC4(C)C3CCC12C]

The bacterium that converts intestinal cholesterol to 4-C-3 can be a naturally occurring bacterium or a recombinant bacterium.

In certain embodiments, the bacterium that converts intestinal cholesterol to 4-C-3 can be a naturally occurring bacterium. Such bacteria may include, but are not limited to, species capable of catalyzing the oxidation of cholesterol via enzymatic pathways involving cholesterol oxidase (CHOX) or 3β-hydroxysteroid dehydrogenase (3β-HSD). Representative examples of bacteria naturally expressing CHOX include Oscillibacter spp. (such as Oscillibacter ruminantium), Rhodococcus spp. (such as Rhodococcus equi, Rhodococcus erythropolis), Streptomyces spp. (such as Streptomyces fradiae, Streptomyces lavendulae), Nocardia spp. (such as Nocardia erythropolis), or any combination thereof. Representative examples of bacteria naturally expressing 3β-HSD include Eubacterium coprostanoligenes, Bacteroides ovatus, Bacteroides vulgatus, or any combination thereof.

In certain embodiments, the bacterium that converts intestinal cholesterol to 4-C-3 can be a recombinant bacterium comprising a nucleic acid encoding CHOX or 3β-HSD, such as the genus Escherichia and genus Bacillus.

In certain embodiments, the bacterium that converts intestinal cholesterol to 4-C-3 is selected from the group consisting of Oscillibacter spp. (such as O. ruminantium), Eubacterium coprostanoligenes, a recombinant bacterium from the genus Escherichia and genus Bacillus comprising a nucleic acid encoding CHOX or 3β-HSD, and any combination thereof. In certain embodiments, the bacterium is O. ruminantium, or a recombinant E. coli bacterium comprising a nucleic acid encoding cholesterol oxidase (CHOX).

In certain embodiments, the recombinant bacterium is selected from the group consisting of an E. coli bacterium, a Bacillus subtilis bacterium, and any combination thereof. In certain embodiments, the recombinant bacterium is selected from the group consisting of E. coli TOP10, E. coli DH5α, E. coli BL21, B. subtilis 168, and any combination thereof. In certain embodiments, the recombinant bacterium comprises a nucleic acid encoding CHOX. B. subtilis 168 can be B. subtilis 168/pMA5-choM1 and B. subtilis 168/pMA5-choM2, method for constructing such bacteria is described in Shao et al. (Shao M, Rao Z, Zhang X, et al. Bioconversion of cholesterol to 4-cholesten-3-one by recombinant Bacillus subtilis expressing choM gene encoding cholesterol oxidase from Mycobacterium neoaurum JC-12[J]. Journal of Chemical Technology & Biotechnology, 2015, 90 (10): 1811-1820.DOI: 10.1002/jctb.4491), which is hereby incorporated by reference in its entirety.

In certain embodiments, the CHOX is selected from the group consisting of CHOX originating from Streptomyces species such as S. fradiae, S. lavendulae, S. cavourensis; and any combination thereof. In certain embodiments, the CHOX is CHOX originating from Streptomyces species such as S. fradiae, S. lavendulae, or S. cavourensis. In certain embodiments, the 3β-HSD originates from a microorganism selected from the group consisting of E. coprostanoligenes, B. ovatus, B. vulgatus, and any combination thereof.

The recombinant bacterium can be constructed by transforming a host bacterium (such as E. coli) with a vector containing the CHOX gene (i.e., a nucleic acid sequence encoding cholesterol oxidase). In certain embodiments, the vector is a plasmid. In certain embodiments, the plasmid is pET (such as pET-21a), pGEX, pBAD, pTrc99A, pUC (such as pUC57), or any combination thereof. In certain embodiments, the plasmid contains a promoter that directs the expression of the CHOX gene in the bacterium. In certain embodiments, the promoter is a constitutive promoter such as J23119. In certain embodiments, the promoter is an inducible promoter such as T7, lac, tac, trc, araBAD, rhaBAD, or P_BAD.

In instances in which 4-C-3 is administered to the subject, the 4-C-3 can be isolated, substantially pure, or both isolated and substantially pure.

In certain embodiments, the bacterium is administered in a therapeutically effective amount to achieve one or more or all of the following:
(i) reduce cholesterol concentrations within tumor tissues, gastrointestinal tract, serum, or feces of the subject;
(ii) increase the 4-C-3 concentrations within tumor tissues, gastrointestinal tract, serum, or feces of the subject;
(iii) inhibit the growth or viability of cancer cells;
(iv) reduce the size, weight, or volume of cancer tumors;
(v) promote apoptosis of cancer cells;
(vi) antagonize the EGFR pathway; and
(vii) target KRAS mutants.

In certain embodiments, the 4-C-3 is administered in a therapeutically effective amount to achieve one or more or all of the following:
(ii) increase the 4-C-3 concentrations within tumor tissues, gastrointestinal tract, serum, or feces of the subject;
(iii) inhibit the growth or viability of cancer cells;
(iv) reduce the size, weight, or volume of tumors;
(v) promote apoptosis of cancer cells;
(vi) antagonize the EGFR pathway; and
(vii) target KRAS mutants.

In certain embodiments, KRAS mutants include but are not limited to G12A, G12C, G12D, G12E, G12F, G12I, G12R, G12S, G12V, G12C G13A, G13C, G13D, G13E, G13R, mutants, either individually or any combination thereof.

In certain embodiments, said cancer cells (such as gastrointestinal tract cancer or lung cancer cells) and/or cancer tumor tissues (such as gastrointestinal tract or lung cancer tumor tissues) contain increased cholesterol concentrations as compared with those from a healthy subject.

In certain embodiments, a fecal sample, a tumor sample, a plasma sample, or a serum sample obtained from the subject prior to administering a therapeutically effective amount of the bacterium or 4-C-3 has at least one of an increased cholesterol concentrations and decreased 4-C-3 concentrations than an average concentration of the cholesterol or 4-C-3 in the corresponding samples obtained from healthy controls (e.g., subjects not suffering from colorectal cancer, lung cancer, liver cancer, or breast cancer).

In certain embodiments, the subject has a lower serum/plasma/fecal/tumor 4-C-3 concentration relative to an average serum/plasma/fecal/tumor 4-C-3 concentration in healthy subjects that do not suffer from a cancer (such as a gastrointestinal tract cancer, e.g., a colorectal cancer or lung cancer). In certain embodiments, the subject has an increased serum/plasma/fecal/tumor cholesterol concentration relative to an average serum/plasma/fecal/tumor cholesterol concentration in healthy subjects that do not suffer from a cancer (such as a gastrointestinal tract cancer, e.g., a colorectal cancer or lung cancer).

In certain embodiments, the method further comprises providing a sample obtained from the subject, wherein the sample is a fecal sample, a tumor sample, a plasm sample, or a serum sample; determining the concentration of at least one of 4-C-3 and cholesterol in the sample; and administering the 4-C-3 or the bacterium to the subject if a decreased concentration of 4-C-3 or an increased concentration of cholesterol is detected in the sample relative to an average concentration detected in healthy subjects.

A tumor sample may include a tumor biopsy specimen.

The decreased concentration of 4-C-3 or an increased concentration of cholesterol in the sample relative to an average concentration detected in healthy subjects can be determined by comparing the measured concentration of 4-C-3 or cholesterol in the sample with the average amount of the concentration of 4-C-3 or cholesterol in samples obtained from healthy controls (e.g., subjects not suffering from colorectal cancer).

The concentration of 4-C-3 and/or cholesterol can be measured using any method known to those skilled in the art. In certain embodiments, the step of determining the concentration of at least one of 4-C-3 and cholesterol in the sample can involve the use of one or more analytical tools, such as high performance liquid chromatography (HPLC), ultra-performance liquid chromatography (UPLC), liquid chromatography mass spectrometry (LCMS), liquid chromatography tandem mass spectrometry (LCMS/MS), gas chromatography-mass spectrometry (GC-MS), or enzymatic colorimetric assays, or combinations thereof. Auxiliary tools such as microscopy, gram staining, polymerase chain reaction (PCR) amplification, sequencing, culture-based screening, or combinations thereof, can be used to characterize microbial populations or metabolic potential related to cholesterol transformation.

In certain embodiments, the method further comprises co-administering one or more additional agents selected from the group consisting of: 5-cholesten-3-one, coprostanone, statins, anti-cancer agents, chemotherapeutic agents, radiation therapeutic agents, immunotherapeutic agents, anti-EGFR therapeutic agents, agents targeting KRAS mutations, and any combination thereof.

In certain embodiments, anti-cancer agents include, but are not limited to chemotherapeutic agents, targeted therapies, immunotherapeutic agents, and combinations thereof. In certain embodiments, chemotherapeutic agents include, but are not limited to, doxorubicin, 5-fluorouracil (5-FU), paclitaxel, cisplatin, cyclophosphamide, or any combination thereof. In certain embodiments, radiation therapeutic agents include, but are not limited to, external beam radiation therapy (EBRT), brachytherapy, and radionuclide-labeled compounds such as iodine-131, lutetium-177, or any combination thereof. In certain embodiments, immunotherapeutic agents include, but are not limited to, immune checkpoint inhibitors (e.g., nivolumab, ipilimumab), chimeric antigen receptor T cells (CAR-T cells), cytokine therapies (e.g., interleukin-2), and monoclonal antibodies (e.g., rituximab, trastuzumab), or any combination thereof.

In certain embodiments, the method further comprising co-administering anti-EGFR therapeutic agents. In certain embodiments, the anti-EGFR therapeutic agent is selected from the group consisting of cetuximab, panitumumab, necitumumab, erlotinib, gefitinib, osimertinib, afatinib, dacomitinib, lapatinib, and any combination thereof.

In certain embodiments, agents targeting KRAS mutations include but are not limited to Adagrasib, Sotorasib, MRTX1133, KRAS G12C inhibitor, or any combination thereof.

In certain embodiments, the method further comprising co-administering anti-colorectal cancer drugs. In certain embodiments, the anti-colorectal cancer drugs are selected from the group consisting of 5-fluorouracil (5-FU), oxaliplatin, irinotecan, capecitabine, cetuximab, panitumumab, bevacizumab, regorafenib, trifluridine/tipiracil, and any combination thereof.

In certain embodiments, the one or more additional agents are administered prior to, concurrently with, or subsequent to the administration of the 4-C-3 or the bacterium.

The cancer (such as gastrointestinal tract cancer, lung cancer, liver cancer, or breast cancer) can be a cancer characterized in high concentrations of cholesterol as compared with a healthy control (e.g., the cancer cell contains high concentrations of cholesterol as compared with a common cell of the same type from a healthy subject).

In certain embodiments, the cancer is selected from the group consisting of a gastrointestinal cancer, a lung cancer, a breast cancer, and any combination thereof.

The gastrointestinal cancer may comprise a gastrointestinal tract cancer, a biliary tract cancer, a liver cancer, or any combination thereof. In certain embodiments, the gastrointestinal tract cancer is selected from the group consisting of esophageal cancer, esophageal adenocarcinoma, gastric cancer, gastric adenocarcinoma, small intestine cancer, gastrointestinal lymphoma, colon cancer, rectal cancer, colorectal cancer, colorectal adenocarcinoma, anal cancer, appendiceal cancer, pancreatic cancer, pancreatic adenocarcinoma, and any combination thereof. In certain embodiments, the gastrointestinal tract cancer is selected from the group consisting of esophageal cancer, gastric cancer, colon cancer, rectal cancer, colorectal cancer, and any combination thereof. In certain embodiments, the gastrointestinal tract cancer is a colorectal cancer.

In certain embodiments, the biliary tract cancer is selected from the group consisting of a gallbladder cancer, a cholangiocarcinoma, and any combination thereof.

In certain embodiments, the breast cancer is selected from the group consisting of hormone receptor-positive breast cancer, triple-negative breast cancer, HER2-enriched breast cancer, and any combination thereof.

In certain embodiments, the lung cancer is selected from the group consisting of non-small cell lung cancer such as lung adenocarcinoma, small cell lung cancer, and any combination thereof.

In certain embodiments, the cancer is a colorectal cancer, a liver cancer, a lung cancer, or a breast cancer.

In certain embodiments, the cancer (such as the gastrointestinal tract cancer, lung cancer, liver cancer, or breast cancer) is characterized by the presence of a KRAS mutation. Such KRAS-mutant cancer can be selected from the group consisting of colorectal cancer, lung cancer, non-small cell lung cancer, pancreatic cancer, liver cancer, breast cancer, and any combination thereof. In certain embodiments, the KRAS mutation is selected from the group consisting of G12A, G12C, G12D, G12E, G12F, G12I, G12R, G12S, G12V, G12C G13A, G13C, G13D, G13E, G13R, and any combination thereof. In certain embodiments, the KRAS mutation is or comprises G12A, G12C, G12D, G12R, G12V, G13D, or a combination thereof.

There are several molecular subtypes of colorectal cancer (CRC), including but not limited to metastatic CRC, KRAS-mutant subtypes (such as G12A, G12D, G12R, G12V, G12C, and G13D), microsatellite instability-high (MSI-H) CRC, and microsatellite stable (MSS) CRC. MSI-H tumors are more likely to respond to immunotherapy, while MSS tumors do not respond as well. Therefore, in certain embodiments, the colorectal cancer mentioned herein includes but is not limited to a metastatic CRC, a KRAS-mutant CRC, an MSI-H CRC, a MSS CRC, or any combination thereof. In instances of KRAS-mutant CRC, the KRAS mutations can be selected from the group consisting of G12A, G12C, G12D, G12R, G12V, G13D, and any combination thereof.

In certain embodiments, the cancer is drug-resistant. In certain embodiments, the cancer is a drug-resistant CRC. In certain embodiments, the CRC is resistant to chemotherapy, targeted therapies, or combinations thereof. In certain embodiments, the CRC is resistant to anti-EGFR therapies (such as cetuximab, panitumumab). In certain embodiments, the CRC comprises resistance-associated mutation(s) selected from the group consisting of KRAS mutation(s) (such as G12A, G12C, G12D, G12R, G12V, G13D), EGFR mutation(s) (such asS492R, G719S), and any combination thereof.

In certain embodiments, the method comprises administering a therapeutically effective amount of: (ai) a bacterium that converts intestinal cholesterol to 4-cholesten-3-one (4-C-3) or (aii) 4-C-3, in combination with (b) an anti-EGFR therapeutic agent to the subject. In certain embodiments, the anti-EGFR therapeutic agent is selected from the group consisting of cetuximab, panitumumab, necitumumab, erlotinib, gefitinib, osimertinib, afatinib, dacomitinib, lapatinib, and any combination thereof. In such cases, the ingredients can be administered simultaneously or separately.

The mode of administration for therapeutic use of the 4-C-3 or bacterium that converts cholesterol to 4-C-3 may be any suitable route that delivers the agent to the subject, such as parenteral administration, e.g., intradermal, intramuscular, intraperitoneal, intravenous or subcutaneous, pulmonary; transmucosal (oral, intranasal, intravaginal, rectal); using a formulation in a tablet, capsule, solution, suspension, powder, gel, particle; and contained in a syringe, an implanted device, osmotic pump, cartridge, micropump; or other means appreciated by the skilled artisan, as well known in the art. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, fecal microbiota transplant, buccal, sublingual, intranasal, or transdermal delivery. In certain embodiments, the 4-C-3 is administered orally. In certain embodiments, the bacterium that converts cholesterol to 4-C-3 is administered orally or by fecal microbiota transplant.

In certain embodiments, the bacterium is provided in the form of a lyophilized powder, a freeze-dried preparation, a microencapsulation, an enteric-coated capsule, a tablet, a granule, a suspension, or any combination thereof. In such cases, excipients (such as a filler, a lubricant, a binder, a disintegrant, a stabilizer, a sweetener, a preservative, a pH adjuster, an anti-caking agent, or the like) can be added to facilitate the formation of the aforementioned dosage forms.

In certain embodiments, administration of 4-C-3 or the bacterium leads to a reduction of tumor (such as CRC tumor) weight by at least 50%, such as 50%, 55%, 60%, 65%, 70, 75%, 80%, or more.

The bacterium, such as the E. coli TOP10 bacterium comprising the nucleic acid encoding CHOX, exhibits high in vivo conversion rate of cholesterol into 4-C-3 upon being administered to the subject. In certain embodiments, the in vivo conversion rate of cholesterol into 4-C-3 is at least 75% (such as 80%, 85%, or more) within 24 hours of administration.

The subject typically refers to humans, but also to other animals, including, e.g., non-human primates, canines, equines, felines, ovines, bovines, porcines, rodents, and the like. In certain embodiments, the subject is a human. In certain embodiments, the subject is a human diagnosed with or at risk of developing colorectal cancer.

Also provided herein is a recombinant E. coli TOP10 bacterium comprising a nucleic acid encoding cholesterol oxidase (CHOX). In certain embodiments, the recombinant E. coli TOP10 bacterium is engineered to express CHOX. In certain embodiments, the nucleic acid is operably linked to a promoter such that the bacterium expresses the CHOX under the control of the promoter.

In certain embodiments, the gene sequence of CHOX comprises or is as set forth in SEQ ID NO: 3. In certain embodiments, the gene sequence of CHOX is at least 80%, such as at least 85%, 90%, 91%, 92%, 93%, 94%, 95%, 96%, 97%, 98%, 99% identical to the sequence set forth in SEQ ID NO: 3. In certain embodiments, SEQ ID NO: 3 comprises the CHOX-encoding sequence. In certain embodiments, the CHOX-encoding sequence corresponds to positions 36 to 1846 of SEQ ID NO: 3. In certain embodiments, the CHOX-encoding sequence is at least 80%, such as at least 85%, 90%, 91%, 92%, 93%, 94%, 95%, 96%, 97%, 98%, 99% identical to the sequence corresponding to positions 36 to 1846 of SEQ ID NO: 3.

In certain embodiments, the promoter is a constitutive promoter such as J23119. In certain embodiments, the promoter is an inducible promoter such as T7, lac, tac, trc, araBAD, rhaBAD, or P_BAD.

Further provided herein is a method of constructing the recombinant E. coli TOP10 bacterium of the above aspects, comprising transforming E. coli TOP10 with a vector containing the CHOX gene. In certain embodiments, the vector is a plasmid. In certain embodiments, the plasmid is pET (such as pET-21a), pGEX, pBAD, pTrc99A, pUC (such as pUC57), or any combination thereof. In certain embodiments, the plasmid contains a promoter (such as J23119) that directs the expression of the CHOX gene in the bacterium.

In certain embodiments, the CHOX is obtained/originated from a microorganism selected from the group consisting of Streptomyces species such as S. fradiae, S. lavendulae, S. cavourensis; and any combination thereof. In certain embodiments, the CHOX is derived from Streptomyces species such as S. fradiae, S. lavendulae, or S. cavourensis.

Further provided herein is a composition comprising the bacterium of any of the above-described embodiments, or 4-C-3. In certain embodiments, the composition is a pharmaceutical composition further comprising a pharmaceutically acceptable carrier, excipient, or diluent.

In certain embodiments, the composition (or pharmaceutical composition) is in the form of a lyophilized powder, a freeze-dried preparation, a microencapsulation, an enteric-coated capsule, a tablet, a granule, a suspension, or any combination thereof.

In certain embodiments, the pharmaceutically acceptable carrier is selected from the group consisting of a filler, a lubricant, a binder, a disintegrant, a stabilizer, a sweetener, a preservative, a pH adjuster, an anti-caking agent, and any combination thereof. In certain embodiments, the pharmaceutically acceptable excipient is selected from the group consisting of a lactose, a mannitol, a PVP, a microcrystalline cellulose, a sodium starch glycolate, a magnesium stearate, a citric acid, an EDTA, a gelatin, and any combination thereof. In certain embodiments, the pharmaceutically acceptable diluent is selected from the group consisting of a saline, a glycerol, a PEG, a sterile water, an ethanol, and any combination thereof.

Also provided herein is a kit comprising the bacterium of any of the above-described embodiments, or 4-C-3, or the composition or pharmaceutical composition as described herein. In certain embodiments, the kit further comprises instructions for use, describing how to administer or apply the bacterium or 4-C-3.

Also provided herein is use of the bacterium of any of the above-described embodiments, or 4-C-3, or the composition or pharmaceutical composition as described herein, in the manufacture of a medicament for treatment of cancers as described herein. Further provided herein is use of the bacterium of any of the above-described embodiments, or 4-C-3, or the composition or pharmaceutical composition as described herein, in the manufacture of a medicament for use in the method of treating a cancer as described herein. The invention also provides the bacterium of any of the above-described embodiments, or 4-C-3, or the composition or pharmaceutical composition as described herein, for use in the treatment of a cancer. In some embodiments, the cancer is a cancer as described herein, for example, a lung cancer or a colorectal cancer (CRC), a liver cancer, or a breast cancer as described herein.

The subject typically refers to humans, but also to other animals, including, e.g., non-human primates, canines, equines, felines, ovines, bovines, porcines, rodents, and the like. In certain embodiments, the subject is a human. In certain embodiments, the subject is a human diagnosed with or at risk of developing colorectal cancer.

As demonstrated in the following experiments, the compound 4-C-3 or the bacterium described herein (such as the E. coli TOP10 bacterium comprising the nucleic acid encoding CHOX) exhibits effective antitumor effect against colorectal and other cancers such as lung cancer. 4-C-3 functions as an endogenous inhibitor of EGFR, exerting its anti-CRC effect primarily through antagonism of the EGFR signaling pathway. It also shows potential to target various KRAS mutants. Due to the dual-targeting of both EGFR and KRAS, 4-C-3 or the bacterium described herein represents an effective therapeutic strategy for overcoming mutant KRAS-driven resistance to anti-EGFR therapies in CRC and other cancers driven by EGFR-KRAS.

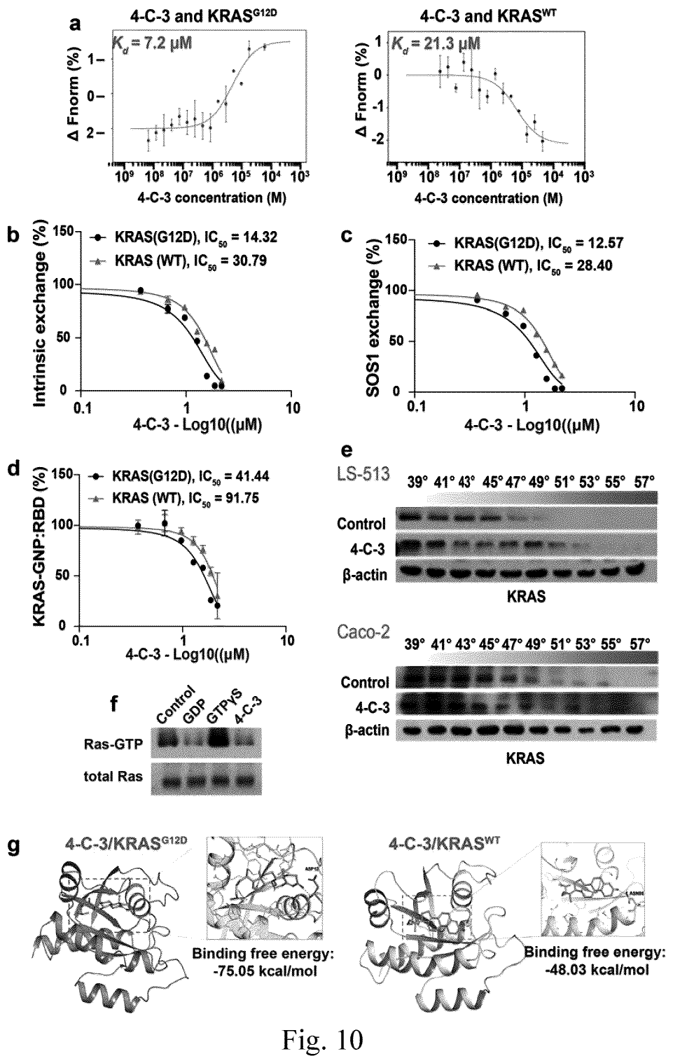

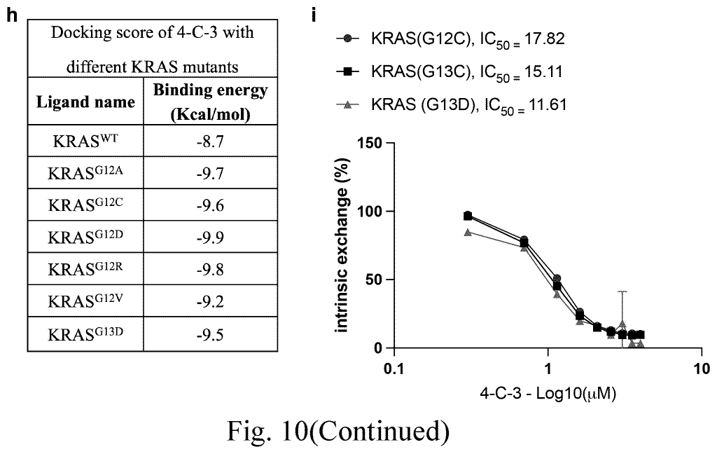

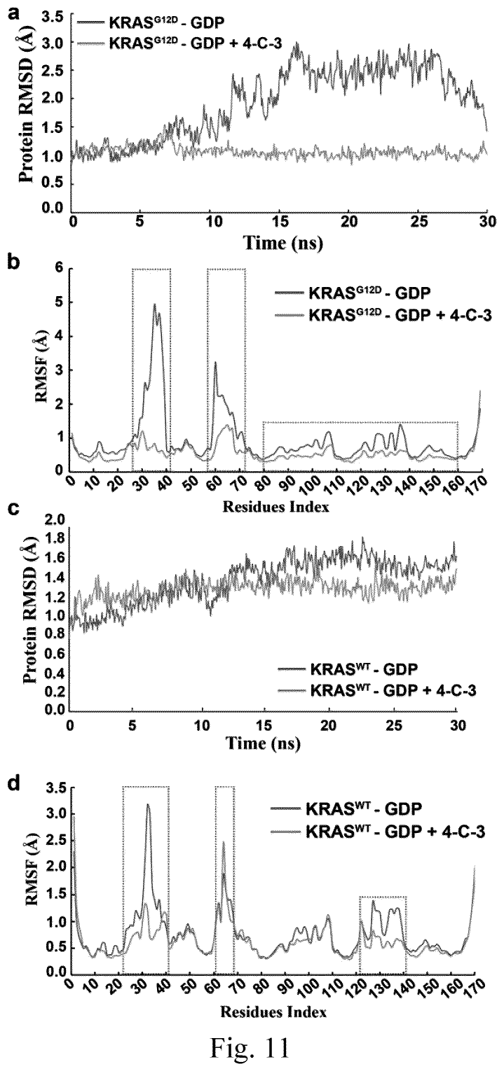

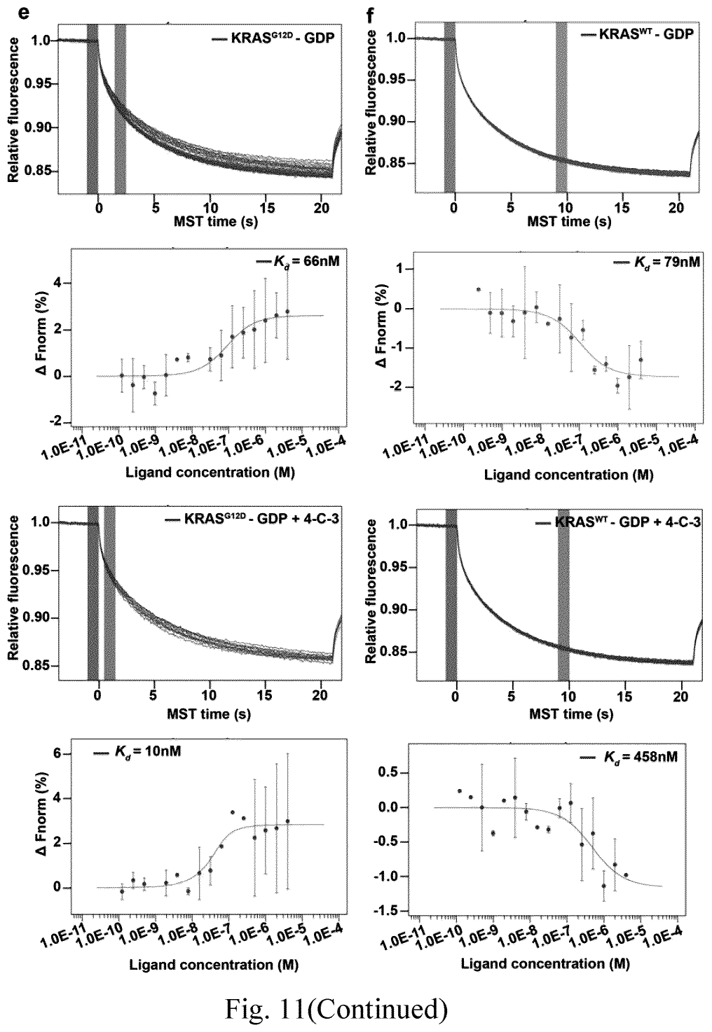

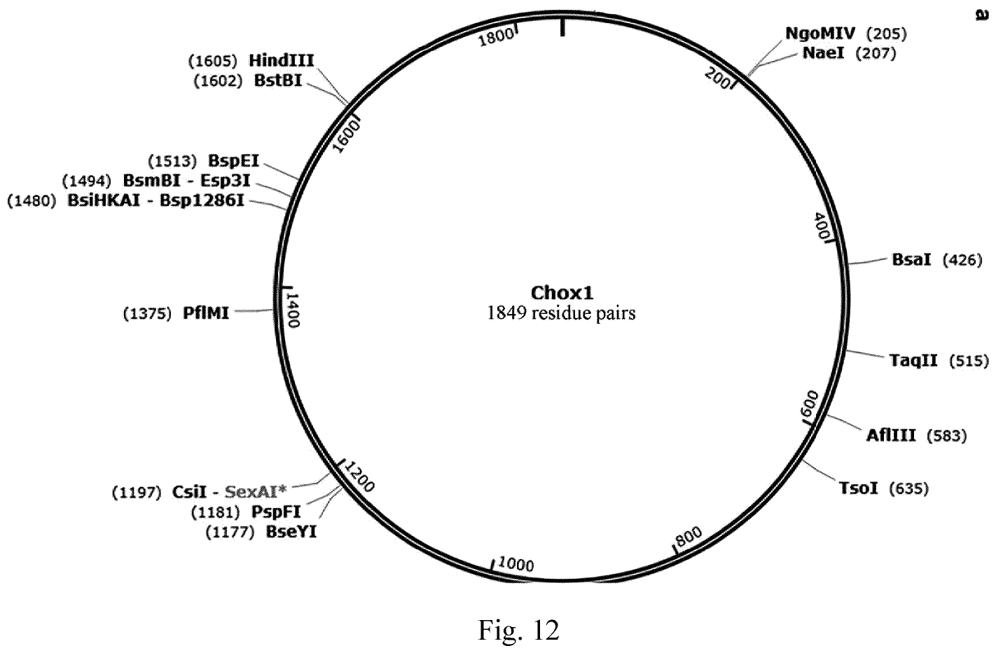

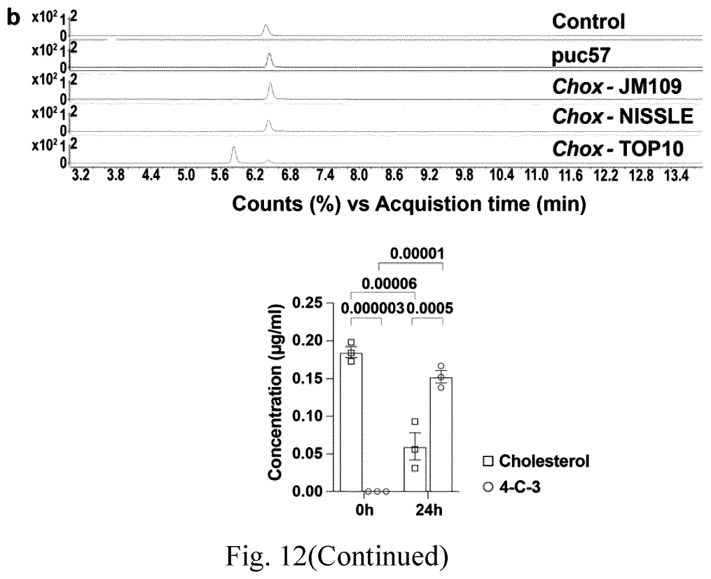

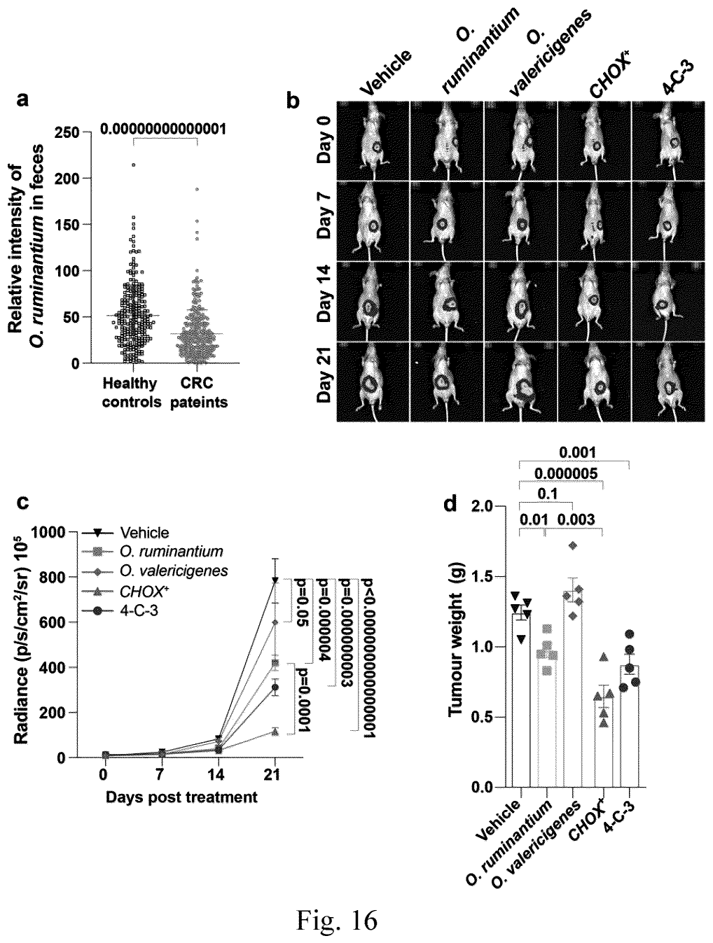

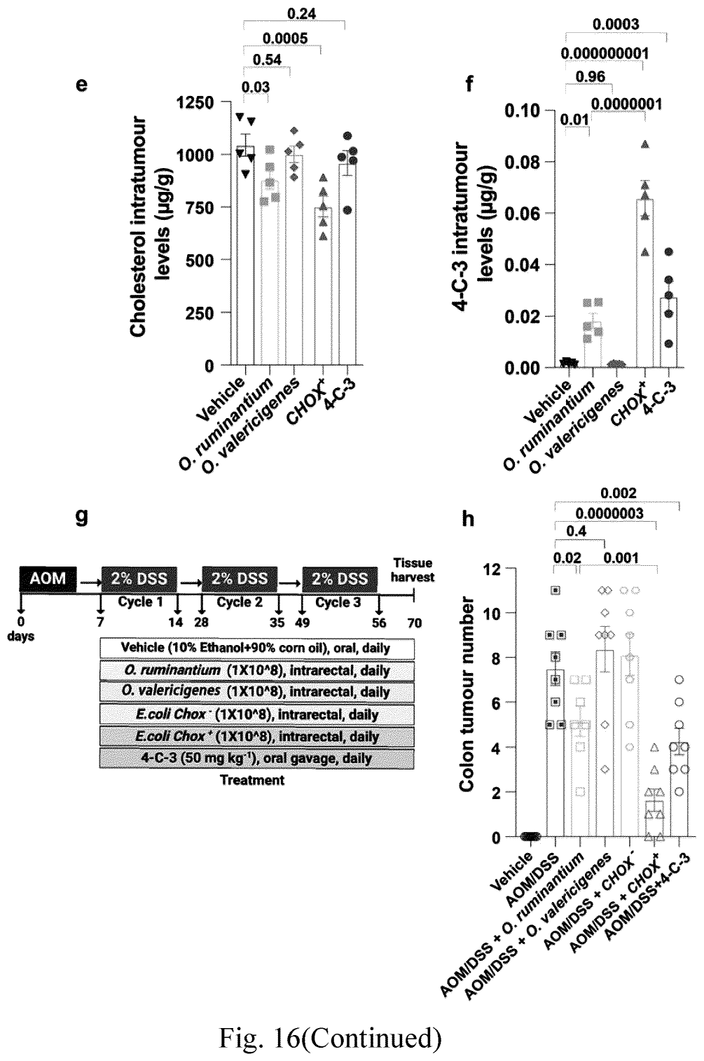

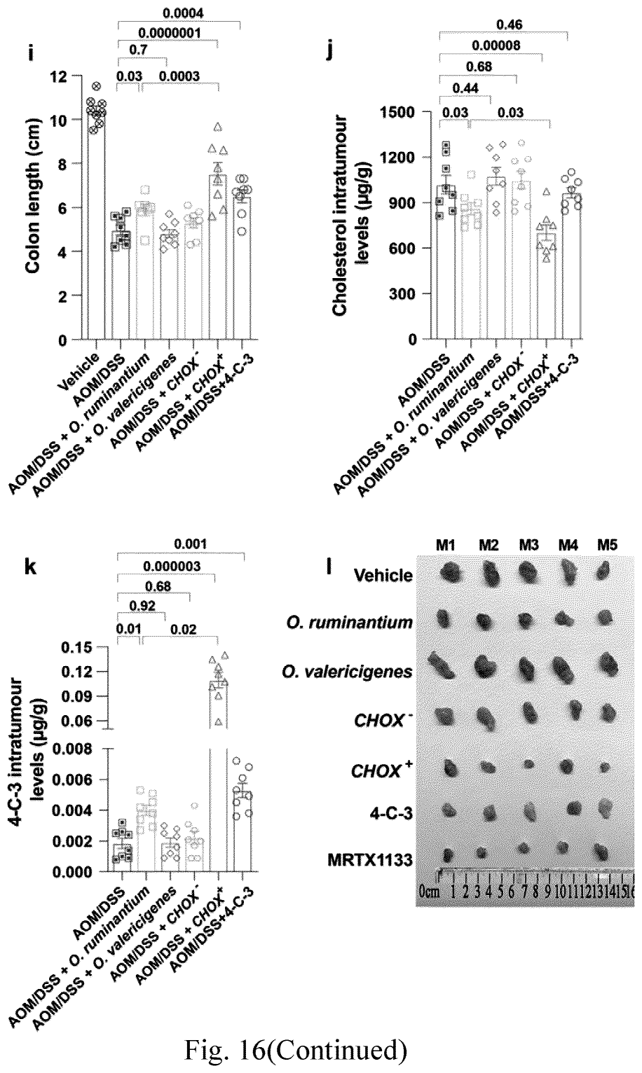

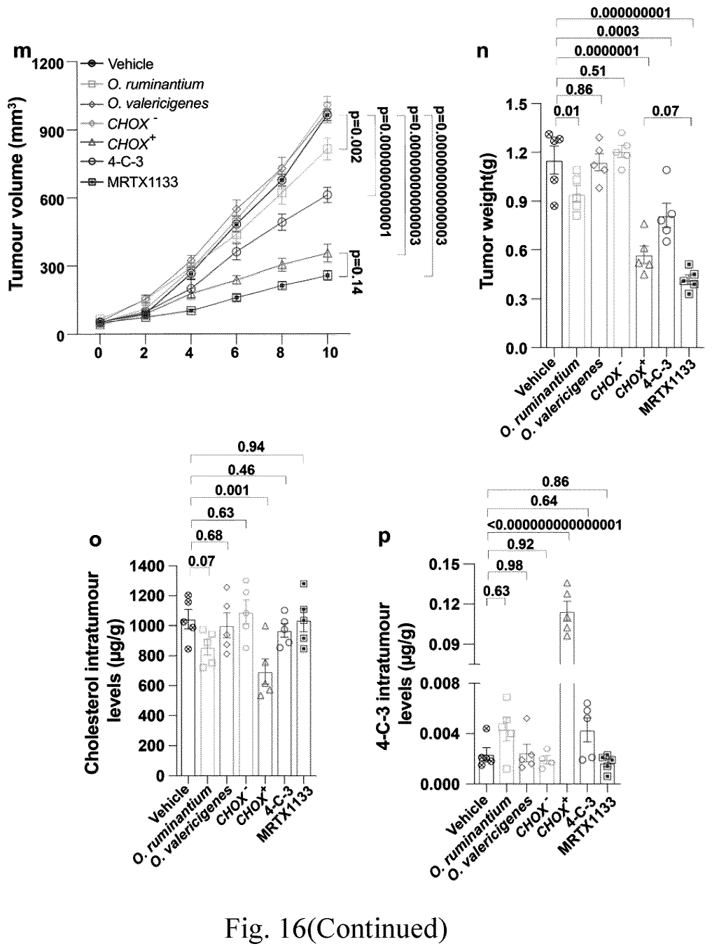

The compound 4-C-3 or the bacterium described herein (such as the E. coli TOP10 bacterium comprising the nucleic acid encoding CHOX) is effective in treating a cancer characterized by the presence of KRAS mutations (such as G12A, G12C, G12D, G12E, G12F, G12I, G12R, G12S, G12V, G12C G13A, G13C, G13D, G13E, G13R). AS verified in the present disclosure, 4-C-3 or CHOX suppresses tumor growth from LS513 (KRAS G12D) and HCT116 (KRAS G13D) xenografts (FIG. 16). Moreover, in fluorescent GDP→GTP exchange assays, 4-C-3 inhibits activation of multiple KRAS mutants (G12C, G13C, G13D) with potency comparable to G12D, indicating broad mutant coverage (FIG. 10). Docking predicts 4-C-3 binds noncovalently in the KRAS switch-II pocket, engaging residue 12 (Asp12/Gly12), a mutational hotspot in G12C/G12D/G13C/G13D; this aligns with the established ligand ability of the KRAS switch-II pocket. In addition, molecular simulations and microscale thermophoresis show 4-C-3 stabilizes KRAS-GDP and inhibits nucleotide exchange, consistent with mechanisms that lock KRAS in its GDP-bound state (FIG. 11).

Moreover, administration of either 4-C-3 or the bacterium involves no obvious side effect on the subject, indicating 4-C-3 and the bacterium will be a safe and feasible therapeutic approach for cancers including CRC. Additionally, the manufacture of both 4-C-3 and the bacterium (such as the E. coli TOP10 bacterium comprising the nucleic acid encoding CHOX) is easily scalable, which is cost-effective in drug development.

### Materials and Methods

### 1. Reagents

TABLE 1
Key reagents or resources

| REAGENT or RESOURCE | SOURCE | IDENTIFIER |
|---|---|---|
| Antibodies | | |
| EREG (D4O5I) Rabbit mAb | Cell Signaling | Cat. #12048 |
| EGF Receptor (E746-A750del Specific) (D6B6) XP ® Rabbit | Cell Signaling | Cat. #2085S |
| mAb | | |
| Phospho-HER2/ErbB2 (Tyr1248)/EGFR (Tyr1173) Antibody | Cell Signaling | Cat. #2244 |
| Phospho-PI3 Kinase p85 (Tyr458)/p55 (Tyr199) (E3U1H) | Cell Signaling | Cat. #17366; |
| Rabbit mAb | | |
| PI3 Kinase p110 δ (D1Q7R) Rabbit mAb | Cell Signaling | Cat. #34050 |
| mTOR (7C10) Rabbit mAb | Cell Signaling | Cat. #2983 |
| Phospho-mTOR (Ser2448) (D9C2) XP ® Rabbit mAb | Cell Signaling | Cat. #5536 |
| Phospho-Akt (Ser473) (D9E) XP ® Rabbit mAb | Cell Signaling | Cat. #4060 |
| K-Ras Antibody | Cell Signaling | Cat. #53270 |
| p44/42 MAPK (Erk1/2) | Cell Signaling | Cat. #4695 |
| Phospho-p44/42 MAPK (Erk1/2) | Cell Signaling | Cat. #9101 |
| Akt Antibody | Cell Signaling | Cat. #9272 |
| β-Actin (D6A8) Rabbit mAb | Cell Signaling | Cat. #8457 |
| Bacterial strains | | |
| Oscillibacter ruminantium | Deutsche | DSM 113516 |
| | Sammlung von | |
| | Mikroorganismen | |
| | und Zullkulturen | |
| Human feces | Clinical trials | N/A |
| Chemicals and reagents | | |
| Cholesterol | SIGMA | CSA. #57-88-5 |
| 5-Cholesten-3-one | SIGMA | CSA. #601-54-7 |
| 4-Cholesten-3-one | MCE | Cat. #HY-113365 |
| Coprostanone | SIGMA | CSA. #566-88-1 |
| Coprostanol | SIGMA | CSA. #360-68-9 |
| Dulbecco's modified Eagle's medium | Gibco | Cat # 11965 |
| Fetal bovine serum | Gibco | Cat # 26140 |
| LB Broth | Thermo Fisher | Cat # 12780052 |
| LB Agar | Thermo Fisher | Cat # 22700025 |
| RIPA buffer | Thermo Fisher | Cat #89900 |
| BCA Protein Assay Kit | Thermo Fisher | Cat #23225 |
| EASYpack Protease Inhibitor Cocktail | Roche | SKU# 5892970001 |
| PhosSTOP | Roche | SKU# 4906845001 |
| Enhanced chemiluminescence | Thermo Fisher | WBKLS0500 |
| TRIzol reagent | Invitrogen | N/A |
| One Step PrimeScript ™ III RT-PCR Kit | TaKaRa | RR600A |
| Quantitect SYBR Green PCR Master Mix | Qiagen, Valencia, | N/A |
| | CA | |
| Cell lines | | |
| HCT116 | ATCC | N/A |
| Colo205 | ATCC | N/A |
| HCT15 | ATCC | N/A |
| Caco-2 | ATCC | N/A |
| DLD-1 | ATCC | N/A |
| HT55 | ATCC | N/A |
| SW620 | ATCC | N/A |
| YAMC | ATCC | N/A |
| LS513 | ATCC | N/A |
| MC38 | ATCC | N/A |
| MCF-7 | ATCC | N/A |
| MDA-MB-231 | ATCC | N/A |
| HCC1806 | ATCC | N/A |
| HepG2 | ATCC | N/A |
| Hep3b | ATCC | N/A |
| HUH7 | ATCC | N/A |
| EO771 | ATCC | N/A |
| LLC | ATCC | N/A |
| Plasmid | | |
| EGFR G719S | Addgene | #11013 |
| pHAGE-EGFR-R222C | Addgene | #116293 |
| pHAGE-EGFR-S492R | Addgene | #116303 |
| pHAGE-EGFR-E114K | Addgene | #116238 |
| pHAGE-EGFR-P596L | Addgene | #116284 |
| Recombinant human protein | | |
| Human EGFR Protein, His Tag | DIMA Biotech | PME100099 |
| Amphiregulin (AREG) (NM_001657) Human Recombinant | OriGene | TP761407 |
| Protein | Technologies | |
| Recombinant Human EREG protein (His tag) | Biosynth | 80R-4058 |
| Human KRAS / K-Ras (G12D & Q61H) Protein (His Tag) | Sino Biological | 12259-H07E1 |
| Human KRAS Protein, His Tag | DIMA Biotech | PME100649 |

### 2. Methods

### 2.1 Cell Cultures

HCT116, Caco-2, SW620, HT55, HT29, Colo205, YAMC, DLD-1, LS513, MC38, and HCT15 cell lines were purchased from ATCC (Manassas, VA, USA). HCT116 cells with stable overexpression of firefly luciferase (Fluc) and enhanced green fluorescent protein (eGFP) were purchased from Imanis Life Sciences. All the cell lines were cultured in DMEM supplemented with 10% fetal bovine serum (FBS) and 1% penicillin/streptomycin at 5% CO₂ in a cell incubator.

### 2.2 MTT Assay

Cell viability was determined by the 3-(4,5-dimethylthiazol-2-yl)-2,5-diphenyltetrazolium bromide (MTT) uptake method. Firstly, the cells (2,000-5,000 per well) were seeded in 96-well plates overnight prior to treatment. The optical density (O.D.) values for the control group were set as 100% viability.

### 2.3 Mouse Models

The mice were purchased from the Animal Center of the Chinese University of Hong Kong and fed in the Laboratory Animal House of Hong Kong Baptist University. The mice were housed under a 12-hour light/dark cycle at a controlled temperature of approximately 25C° and humidity (˜60%) with free access to food and water. The mice were fed standard laboratory chow diets for one week before starting the experiment to allow for adaptation. The procedures for all in vivo studies were approved by the Committee on the Use of Human & Animal Subjects in Teaching & Research (HASC) at Hong Kong Baptist University and procedures were approved by the Department of Health under Hong Kong legislation.

Subcutaneous mouse models: six-week-old male BALB/c nude mice or C57BL/6 mice were subcutaneously inoculated with 5×10⁶ DLD-1 cells, 1×10⁶ HCT116 cells, 2×10⁶ LS513 cells, or 1×10⁶ MC38 cells in the left armpit. For breast cancer and lung cancer mode models, six-week-old male C57BL/6 mice were subcutaneously inoculated with 1×10⁶ EO771 or LLC cells into both flanks. Once tumors were palpable (˜60 mm³), the tumor-bearing nude mice were randomly assigned for different treatments. At the end of the experiment, tumor dimensions were measured with a caliper, and tumor volume was calculated with the formula: (L×W²)/2, where L and W represent the length and width, respectively, with diameter in mm.

Orthotopic mouse models: athymic Balb/c mice aged 5-6 weeks were maintained in laminar-flow cabinets with free access to standard chow diets and sterile water. The orthotopic CRC tumor model was constructed as described in Hu X et al. (Hu X et al., “Dihydroartemisinin is potential therapeutics for treating late-stage CRC by targeting the elevated c-myc level.” Cell Death & Disease. 2021 Nov. 5; 12(11):1053). Briefly, mice were anesthetized and underwent laparotomy with the cecum exposed and exteriorized. HCT116-Fluc-Neo/GFP-puro cells (5×10⁵) were suspended in 40 μl of PBS/Matrigel (1:1) and inoculated into the submucosa of the mice cecum with a 29-gauge needle. One week after cell inoculation, the mice were randomized into four groups. Mice in the control group were treated with vehicle (10% ethanol+90% corn oil), and mice in the treatment group were given 10 mg/kg, 25 mg/kg, and 50 mg/kg 4-C-3. Bioluminescence signal was monitored using the IVIS 2000 Imaging System coupled with Living Imaging Software (Caliper). D-luciferin substrate (150 mg/kg) was intraperitoneally injected into mice 10 minutes before the bioluminescence signal measurement. No blinding was done. Mice were sacrificed 24 days after the treatment, and the tumors were dissected and fixed in 4% paraformaldehyde for further analyses.

Patient-derived xenograft (PDX) models: Human colorectal tumor specimens (P=0) were collected from surgical patients at the First People's Hospital of Huizhou. Samples were preserved during transit to the laboratory by immersion in 50 ml centrifuge tubes containing saline with 10% penicillin-streptomycin and placed on ice. The cancer tissue specimens were thoroughly rinsed three times with a large amount of physiological saline containing 10% gentamicin sulfate to remove residual impurities from the tissue block. Tumor tissue was cut into pieces approximately 2× 2× 2 mm³ in size using a sterile scalpel and forceps. A puncture inoculation needle was utilized for subcutaneous inoculation, and the tumor tissue was implanted into the axillary area of the forelimb of a nude mouse. Then, the PDX model with growing P1 generation tumor cells was established. Tumor volume was calculated twice weekly using the formula: (L×W²)/2, where L and W represent the length and width, respectively, with diameter in mm. When the P1 generation tumor grew to about 1000 mm³, the mice were euthanized, and the tumor surface mucous and connective tissues were removed under ice bath conditions. Next, the tumor mass was cut open, and the necrotic part was removed. The processed P1 generation tumor tissue was then inoculated into the axilla of 10 male nude mice aged 7-8 weeks and weighing 19±1 g, according to the above tumor inoculation method. The formal experiment was conducted using PDX tumor tissues at P3 generation.

AOM/DSS models: Mice were randomly divided into four experimental groups and one control group (n=8 per group). The experimental groups were intraperitoneally injected with a single dose of 10 mg/kg azoxymethane (AOM) on the first day of the first week, then fed with drinking water supplemented with 2% Dextran Sodium Sulfate (DSS) for 1 week, followed by normal drinking water for 2 weeks, with 3 weeks as a cycle, which was repeated three times. In addition, mice in the 4-C-3 treatment group were orally administrated with 4-C-3 at the dosage of 50 mg/kg/day/mouse, and the treatment group with engineered bacteria were orally administrated with E. coli CHOX/E. coli CHOX-bacteria (1×10⁸ CFU/day/mouse). In the control group, mice were treated with the same amount of PBS. At the end of the experiment, the mice were euthanized and the colons were opened longitudinally. The visible tumors were counted, and tumor dimensions were measured with a caliper. Tumor volume was calculated with the formula: (L×W²)/2, where L and W represent the length and width, respectively, with diameter in mm. The tumor burden was calculated by summing the volumes of all tumors.

### 2.4 Human Specimens

Fresh human colon cancer specimens were collected from CRC patients after surgical tumor resection at the First People's Hospital of Huizhou. None of the patients had received chemotherapy or immunosuppressive therapy for at least 3 months prior to the surgery. To obtain the normal tissue, a distance of more than 5 cm to the tumors was kept. The use of tumor tissue samples for research purposes was approved by the Institutional Human Research Ethics Committee at the First People's Hospital of Huizhou. This study was performed in accordance with the Declaration of Helsinki.

### 2.5 Patient-Derived Organoids

The isolation of patient-derived organoids was performed as our previously published study (Su, Anthony Chin Yang et al., “Lactococcus lactis HkyuLL 10 suppresses colorectal tumorigenesis and restores gut microbiota through its generated alpha-mannosidase.” Gut 73, no. 9 (2024): 1478-1488). Briefly, fresh human colon cancer specimens were collected from CRC patients after surgical tumor resection at the First People's Hospital of Huizhou. The colonic tissues were washed thoroughly with ice-cold saline containing antibiotics and antifungals, and the underlying muscle layer was removed from the submucosal layer. The tissues were cut into fine pieces that were then incubated in a solution of collagenase and/or Dispase at 37° C. with gentle agitation until the tissue is dissociated into single cells or small clusters. After isolation, cells were suspended in the matrix composed of advanced DMEM/F12 medium (Gibco) and growth-factor-reduced Matrigel in a ratio of 1:1. The cell-matrix mixture was seeded in 48-well plates (1000 single cells per 25 μl of Matrigel per well). The Matrigel was polymerized for 10 min at 37° C. and 250 μl of the culture medium consisting of advanced DMEM/F12 medium+Glutamax (Invitrogen) containing N2, B27 supplements (Invitrogen), 10 mmol/L HEPES (Invitrogen), 1.25 mmol/L N-acetyl cysteine (Sigma-Aldrich), 2 mmol/L glutamine, 10 μmol/L R-spondin-1, Noggin, WNT3A, SB202190 (Sigma-Aldrich), 50 ng/ml EGF (Invitrogen), and 50 μg/mL gentamycin. We developed five concentration gradients for the in vitro test. The PDO samples were treated with 4-C-3 for 5 days, and during the treatment period, the growth of the organoids was observed in a light microscope. At least 5 random fields were taken for quantification. The effect of the drugs was verified by observing the growth, activity, morphology, and cell clusters of the organoids.

### 2.6 Western Blot Analysis

The experiment was performed as described in our previous studies [Chow, C. F. W., et al. (2022). Body weight regulation via MT1-MMP-mediated cleavage of GFRAL. Nat Metab 4, 203-212; Guo, X., et al. (2024). Artesunate treats obesity in male mice and non-human primates through GDF15/GFRAL signalling axis. Nat Commun 15, 1034]. After the treatments, the whole-cell lysates of the CRC cells were collected by suspending the cells in RIPA lysis buffer, then centrifuging the samples at 14,000 rpm for 10 minutes at 4° C. The protein concentration was measured and calculated using a BCA Protein Assay Kit. Depending on the detection marker, 10 to 25 μg of proteins were loaded into 10% SDS-PAGE and then transferred to PVDF membranes. After transfer, the membranes were incubated in blocking solution (5% skim milk powder in TBS containing Tween 20) for 1 hour at room temperature, and then the membranes were incubated with EGFR, pEGFR, PI3 Kinase p85/p55, PI3 Kinase p110δ, mTOR, Phospho-mTOR, Phospho-Akt, K-Ras, p44/42 MAPK (Erk1/2), Phospho-p44/42 MAPK (Erk1/2), Akt, β-Actin respectively overnight at 4° C. After that, the membranes were incubated with anti-rabbit IgG antibody for 1 hour at room temperature. All antibody solutions were diluted in TBS containing Tween 20 and 5% dry milk. The membranes were exposed using enhanced X-ray film and ECL reagent.

### 2.7 Real Time PCR

The experiment was performed as described in our previous studies [Guo, X., et al. (2022a). Regulation of age-associated insulin resistance by MT1-MMP-mediated cleavage of insulin receptor. Nat Commun 13, 3749; Guo, X., et al. (2022b). Control of SARS-COV-2 infection by MT1-MMP-mediated shedding of ACE2. Nat Commun 13, 7907]. Total RNA was extracted from samples using TRIzol reagent (Invitrogen) in accordance with the RNA extraction protocol of Thermo Fisher Scientific. cDNAs were subsequently prepared using the One Step PrimeScript™ III RT-PCR Kit (RR600A, TaKaRa). Real-time polymerase chain reaction (PCR) was performed using the Quantitect SYBR Green PCR Master Mix (Qiagen, Valencia, CA) with 1 μL cDNA in a final volume of 10 μL and the following primers at a final concentration of 1000 nM. Primers for EREG were 5′-ACGTGTGGCTCAAGTGTCAA-3′ (forward; SEQ ID NO: 1) and 5′-CACTTCACACCTGCAGTAGTTT-3′ (reverse; SEQ ID NO: 2). Amplification was performed using the LightCycler 2000 instrument (Roche, Indianapolis, IN). The cycling conditions comprised a denaturation step for 15 minutes at 95° C., followed by 40 cycles of denaturation (95° C. for 15 seconds), annealing (59° C. for 20 seconds), and extension (72° C. for 15 seconds). After amplification, a melting curve analysis was performed with denaturation at 95° C. for 5 seconds, then continuous fluorescence measurement was made from 70° C. to 95° C. at 0.1° C./second.

### 2.8 Flow Cytometer Analysis

Flow cytometry analysis was performed to determine cell apoptosis. Treated cells were harvested using trypsin without EDTA and phenol red to prevent any interference with the staining process. After harvesting, the cells were washed twice with cold PBS to remove any residual trypsin and other contaminants. Subsequently, 100 μL of the cell suspension was transferred to a 5 mL culture tube. Cells were then incubated with 5 μL of FITC-conjugated annexin-V reagent (2.5 mg/mL) and 5 μL of propidium iodide (PI) solution (5 mg/mL) for 15 minutes at room temperature in the dark. After incubation, 400 μL of binding buffer was added to each tube. The stained cells were immediately analyzed using a flow cytometer equipped with appropriate filters for FITC and PI detection. Data was acquired and analyzed using FlowJo software to determine the percentage of apoptotic cells.

### 2.9 Human Study

522 fecal samples were collected from the Fudan University Shanghai Cancer Center, Shanghai, China, and the Second Hospital of Shandong University, Shandong, China, from 2018 to 2021. Patients were diagnosed with CRC following postoperative pathological examination of tissue biopsies collected during colonoscopy. Fecal samples were obtained from patients at the hospital approximately two weeks after colonoscopy and stored at −80° C. before undergoing metagenomic sequencing and metabolomics profiling. Participants' demographics and clinicopathological characteristics, including age, gender, tumor location, size, differentiation, TNM stage, KRAS/NRAS/BRAF mutation, nerve invasion, lymphatic invasion, vascular invasion, MMR status, administration of neoadjuvant therapy, administration of adjuvant therapy, history of diabetes, hypertension, and other cardiovascular diseases, were collected from the electronic medical record system. The healthy controls were recruited volunteers with no gastrointestinal tumors confirmed by colonoscopy screening. Healthy controls were excluded if they had a history of enteric disorders including chronic diarrhea, inflammatory bowel disease, or gastroesophageal reflux disease; previous cardiovascular disease; diabetes mellitus; previous other malignancies; antibiotic use within six months before enrollment; or other infectious diseases.

### 2.10 Mass Spectrometry Analysis

An AGILENT ultra-performance liquid chromatography (UPLC) system coupled to a triple quadrupole (QQQ) MS6460 mass spectrometer was used for the targeted metabolomics profiling study. A Waters BEH 2.1×50 mm C18 1.7 μm column with a pre-column was used. The mobile phase used in LC-MS-QQQ was A: water and B: MeOH. The gradients were set as 80% B (0-10 min), 100% B (10-12 min), 80% B (12-12.1 min), 80% B (12.1-14 min). In targeted metabolomics, the MS data was collected and processed by in-house software provided by Agilent.

Approximately 80 mg of tumor tissue or adjacent normal tissue samples were mixed with 1 mL of methanol after adding steel beads. The mixture was vigorously vortexed and then incubated overnight at 4° C. Following this, the mixture was centrifuged at 15,000 rpm for 10 minutes at 4° C. The supernatant (200 μL) was collected for LC-MS analysis.

### 2.11 Bacteria Strains

Oscillibacter ruminantium (DSM113516) were cultured in Medium 104b under anaerobic conditions at 37° C. The Chox gene from Streptomyces species was cloned into the pUC57 vector using standard molecular cloning techniques, including restriction digestion and ligation. The resulting plasmid was transformed into E. coli (TOP10) by heat shock transformation. The insertion of the Chox gene into E. coli was confirmed by PCR using gene-specific primers and agarose gel electrophoresis. Additionally, a pUC57 vector-only control strain of E. coli was constructed by transforming E. coli with the empty pUC57 vector. Both the Chox-expressing E. coli and the vector-only control strains were incubated in LB medium supplemented with 100 μg/mL ampicillin and 0.5% cholesterol at 37° C. for 24 hours with shaking at 200 rpm to ensure proper aeration and nutrient distribution. Post-incubation, the culture broth was centrifuged at 5,000 r.p.m. for 10 minutes to pellet the bacterial cells. The supernatant was collected, and the cholesterol and its metabolites in the culture broth were extracted using a liquid-liquid extraction method with chloroform and methanol. The extracted metabolites were then dried under nitrogen gas, reconstituted in a suitable solvent (e.g., methanol), and quantified using LC-MS. The LC-MS analysis was performed on an Agilent 1290 Infinity II coupled with an Agilent 6460 Triple Quadrupole mass spectrometer, using a C18 reverse-phase column (2.1×50 mm, 1.7 μm particle size) with a binary solvent system consisting of water (solvent A) and acetonitrile (solvent B) containing 0.1% formic acid. The gradient elution was set from 10% B to 90% B over 15 minutes, with a flow rate of 0.3 mL/min. Quantification was achieved by comparing the retention times and mass spectra of the samples to those of cholesterol and its known metabolites.

### 2.12 Cellular Thermal Shift Assay

Cells treated with 4-C-3 were disrupted by liquid nitrogen, then centrifuged at 12,000 r.p.m. for 15 min to obtain total protein extracts. The proteins were divided into ten fractions and denatured at different temperatures for 10 min. Denatured proteins were precipitated by centrifugation at 12,000 r.p.m. for 10 min. EGFR and KRAS were detected by Western blot.

### 2.13 Microscale Thermophoresis

Recombinant human protein was labeled with a Monolith NT Protein Labeling Kit RED-NHS (Nanotemper, Munich, Germany) and diluted to 50 nM with HEPES buffer (10 mM HEPES, 150 mM NaCl, 0.05% Tween). 4-C-3 was diluted with 1% ethanol in HEPES buffer to concentrations ranging from 125 μM to 3.8 nM. Samples were incubated at room temperature for 5 minutes. The mixtures were centrifuged at 16,000×g for 5 minutes and loaded into capillaries. Microscale thermophoresis measurements were performed using a Monolith NT.115 (Nanotemper, Munich, Germany).

### 2.14 Ras Activation Assay

The Ras activation assay was conducted using a Pan-Ras Activation Kit (Cell Biolabs) following the manufacturer's protocol. For control samples, cell lysates were treated with GTPγS and GDP at 30° C. for 30 minutes, then MgCl₂ was added. The lysates were then mixed with Raf1-RBD GST-conjugated agarose beads for 1 hour at 4° C., followed by washing. The proteins bound to Raf1-RBD were eluted using 2× sample loading buffer, and the co-precipitated Ras protein was analyzed via Western blotting.

### 2.15 Molecular Dynamics Simulation

Molecular dynamics simulations were conducted using Schrödinger software and the OPLS 2005 force field. The initial dimensions of the periodic box were 10 nm×10 nm×10 nm. The necessary number of counter ions (sodium cations) were added to ensure the electric neutrality of the system. The simulation was performed with periodic boundary conditions in all three directions, using a 100 ps timestep. The energy of the system was initially minimized using the steepest descent algorithm. Equilibrations were carried out in two steps. First, an NVT (constant Number of atoms, Volume, and Temperature) simulation was performed to bring the system to the target temperature. Second, an NPT (constant Number of atoms, Pressure, and Temperature) simulation was performed. Each ensemble was used to stabilize the temperature and pressure at 300 K and 1.01 bar, respectively. During the production dynamics, the structure was simulated for 100 ps using the NPT ensemble. Finally, the simulation was run for 30 ns.

### 2.16 Docking

The molecular docking was performed using the Glide module in Schrödinger. The binding site was selected as the center of the internal ligand in the crystal structure. The docking was performed with standard precision (SP-docking), and the GlideScore built into Schrödinger was used as the scoring function. For each docking simulation, 10 conformations were generated for each small molecule, and their energies were minimized after docking. For each conformation generated by docking small molecules, the low-energy binding conformation was selected for analysis.

### 2.17 Quantification and Statistical Analysis

All the results were obtained from multiple experiments (at least three independent experiments). Data were expressed as average with SD or SEM values where appropriate. Significance p-values were calculated using GraphPad Prism 8, and p-values less than 0.05 were regarded as statistically significant. Spearman's rank coefficient correlation analysis was used for metabolite quantity correlation and the correlation between cholesterol and bacteria derived from patients with CRC. The Wilcoxon rank-sum two-tailed test was used to determine metabolite differences between CRC and healthy participants. The Wilcoxon one-tailed test was used to determine whether CRC-associated gut microbiota produces lower levels of cholesterol metabolites in the metagenomic data. Unpaired t-tests or one-way ANOVA were used in other experiments as indicated in the figure legends.

### Results

### 1. The Level of 4-C-3 Reduced in Human Colon Cancer

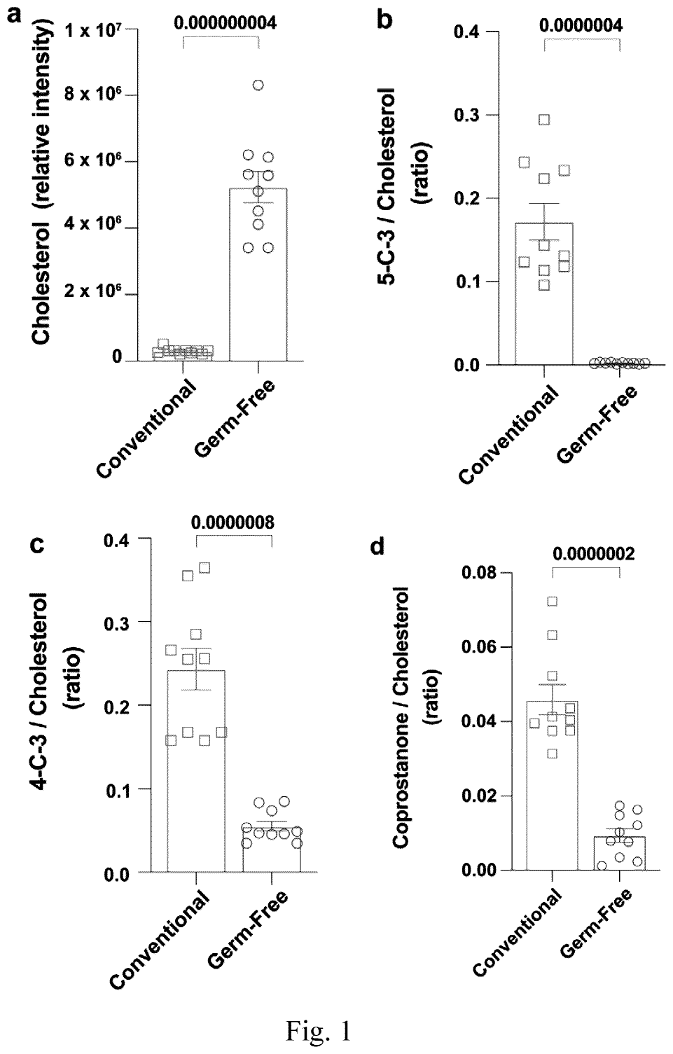

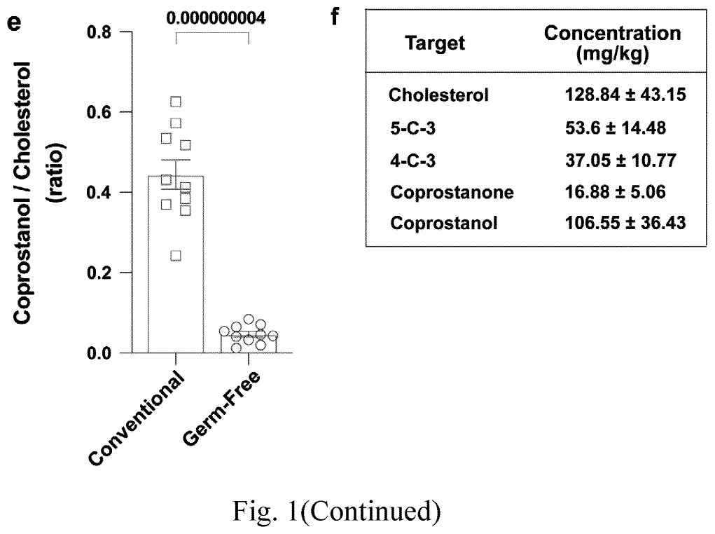

To address the potential contributions of microbial metabolites of intestinal cholesterol to the development of CRC, we first collected fecal samples from germ-free mice and then detected the contents of microbial products associated with cholesterol metabolism, including 4-C-3, 5-C-3, coprostanone, and coprostanol. The results show that fecal cholesterol was significantly enriched in germ-free mice compared with conventional mice (FIG. 1a). However, the above cholesterol metabolites were largely depleted in germ-free mice (FIG. 1b-e), confirming the contribution of the gut microbiota to cholesterol metabolism in the gut.

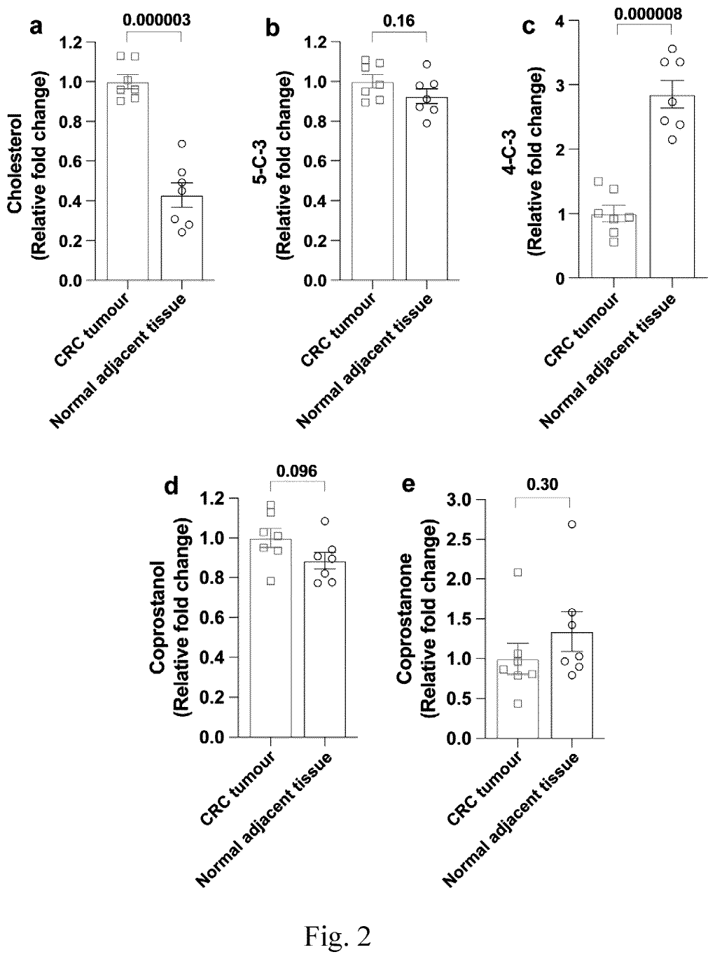

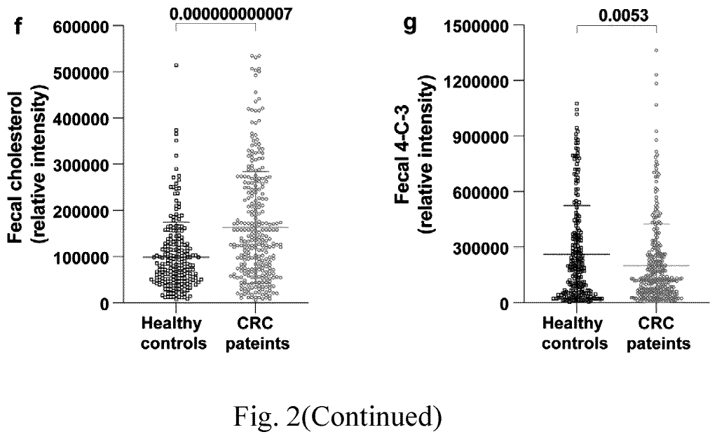

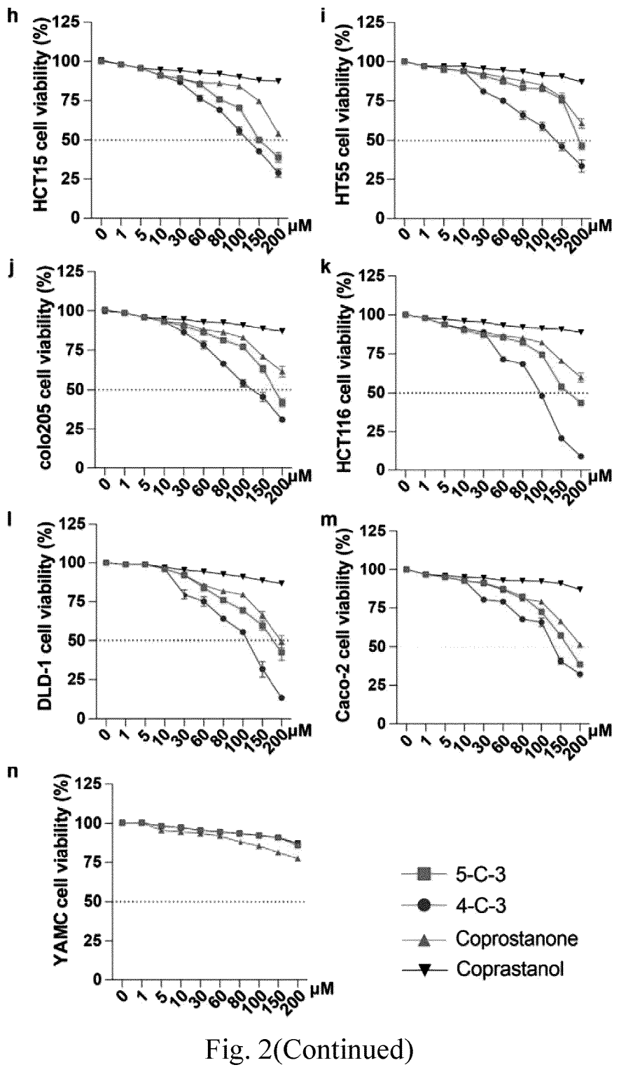

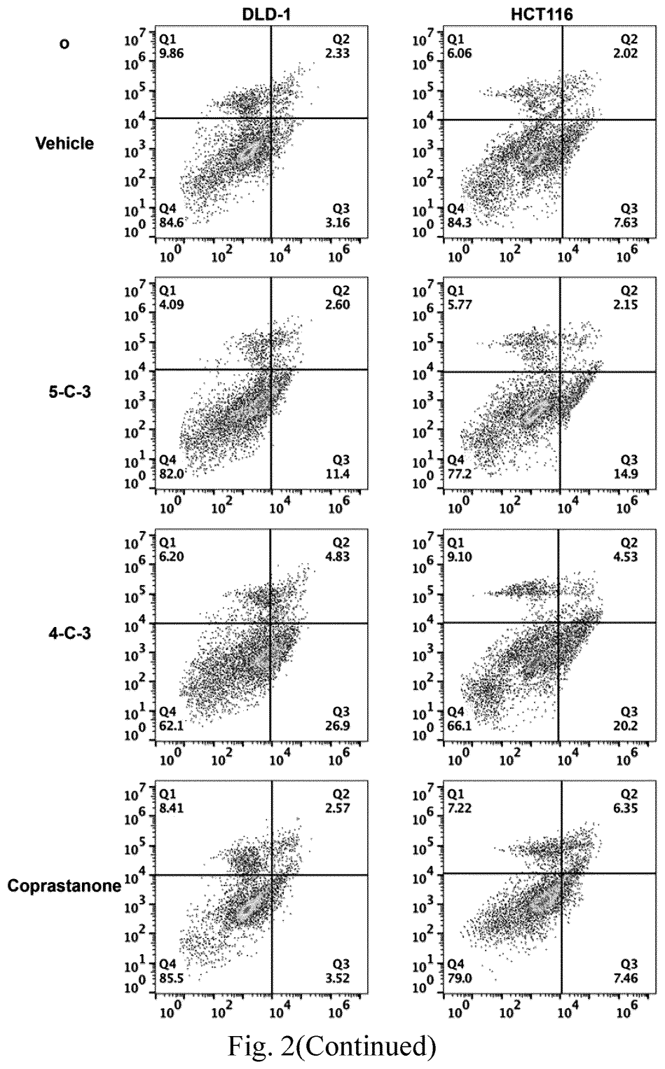

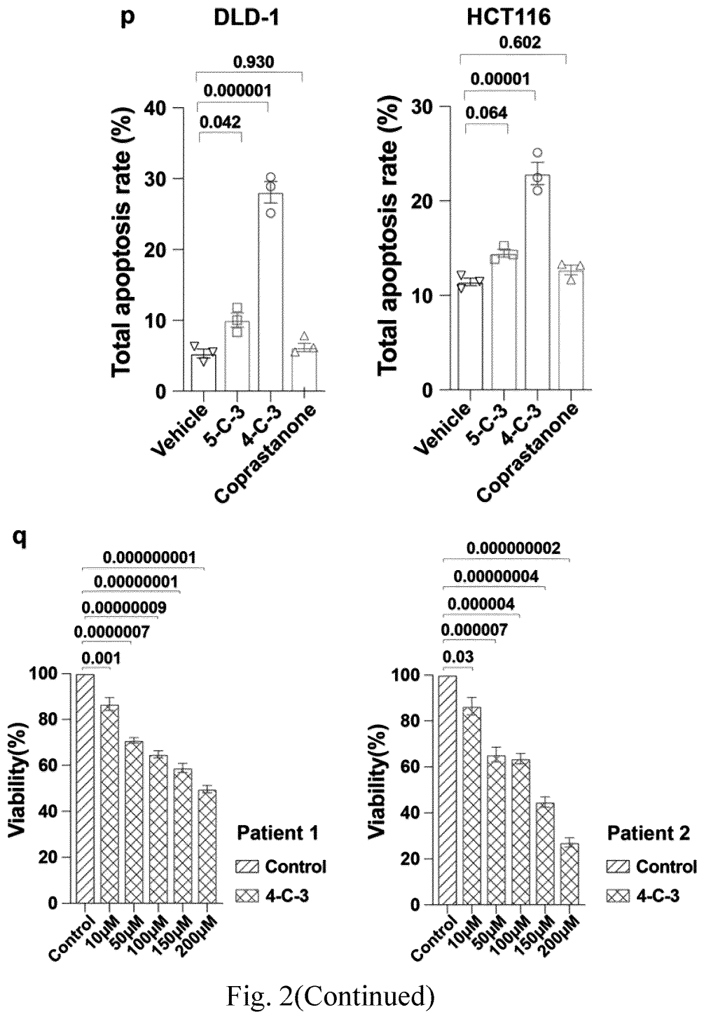

To evaluate the clinical relevance of our findings obtained from mouse studies, the levels of cholesterol and its microbial metabolites were investigated in patients with CRC. Increased cholesterol level was observed in CRC tumor biopsies compared with that in adjacent normal tissues (FIG. 2a). Among the major microbial metabolic products of cholesterol investigated in this study (FIG. 2b-e), we found that 4-C-3 was specifically depleted in tumor biopsies (FIG. 2c). We next examined fecal cholesterol and 4-C-3 levels in a CRC patient cohort with 532 samples reported in our previous studies [Kong, C., et al. (2023). Integrated metagenomic and metabolomic analysis reveals distinct gut-microbiome-derived phenotypes in early-onset colorectal cancer. Gut 72, 1129-1142; Li, J., et al. (2024b). Microbial and metabolic profiles unveil mutualistic microbe-microbe interaction in obesity-related colorectal cancer. Cell Rep Med 5, 101429; Yang, Y., et al. (2021). Dysbiosis of human gut microbiome in young-onset colorectal cancer. Nat Commun 12, 6757]. Increased fecal cholesterol levels (FIG. 2f) and reduced fecal 4-C-3 levels (FIG. 2g) were observed in CRC patients compared with healthy controls.

### 2. 4-C-3 Protects Against Colorectal Tumorigenesis In Vitro and In Vivo

We next investigated the effects of these metabolites in a panel of CRC cell lines, including HCT15, HT55, Colo205, HCT116, DLD-1 and Caco-2 (FIG. 2h-m). We found that all of these metabolites, except coprostanol, exerted different degrees of inhibitory effects on the growth of CRC cell lines in a dose-dependent manner, whereas almost no change in growth was observed in the noncancerous colon cell line YAMC (FIG. 2n), suggesting that the growth-suppressive effects of these metabolites preferentially affect cancer cells. Among the metabolites investigated, 4-C-3, at dosages within the physiological range found in fecal samples of normal human subjects, exhibited the most potent anti-proliferative effect in CRC cell lines (FIG. 1f; FIG. 2h-m). Consistently, flow cytometry analyses revealed that among the microbial products, 4-C-3 led to the greatest percentage of apoptotic cells in CRC cell lines (FIG. 20-p). The potent anti-CRC effect of 4-C-3 was further confirmed in cancer organoids derived from two CRC patients (FIG. 2q). Given the robust anti-CRC effect of 4-C-3 in vitro and ex vivo, we focused mainly on 4-C-3 in the following in vivo investigations.

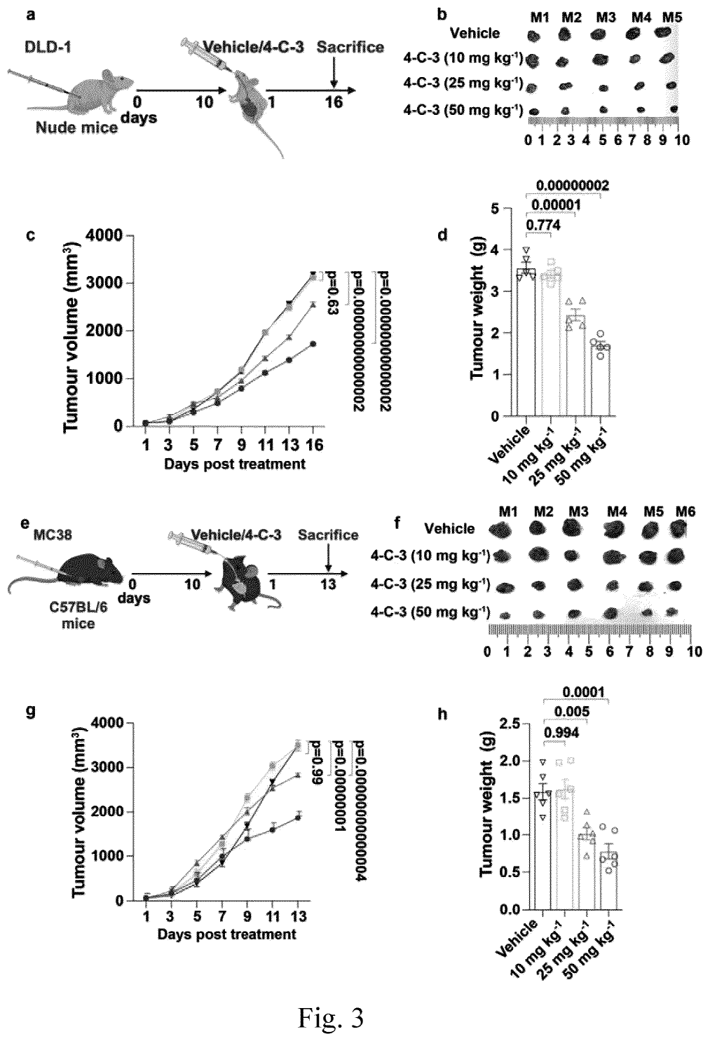

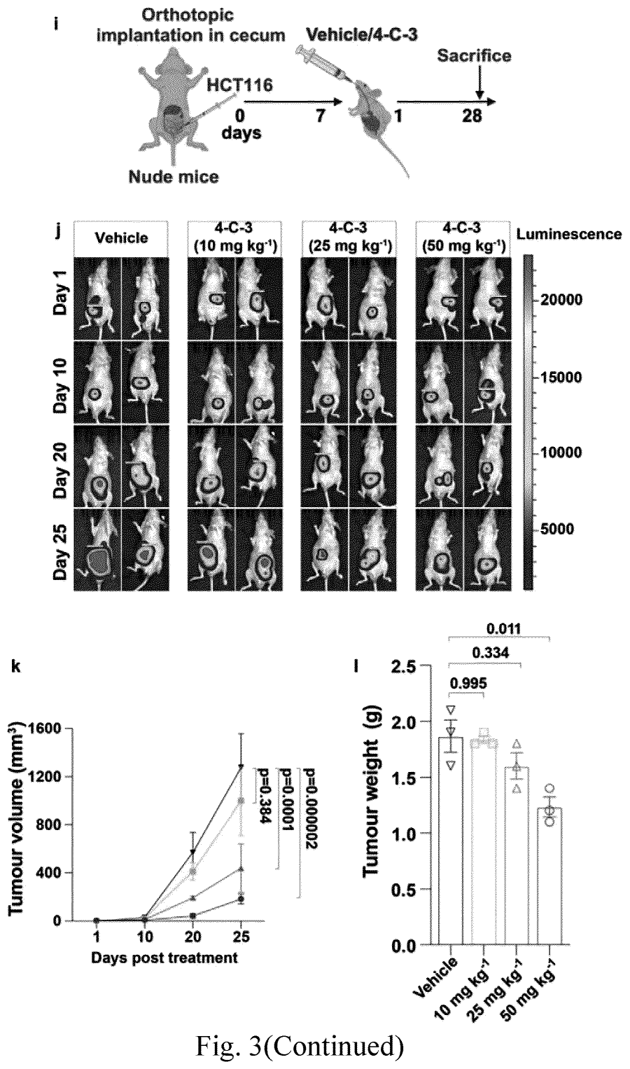

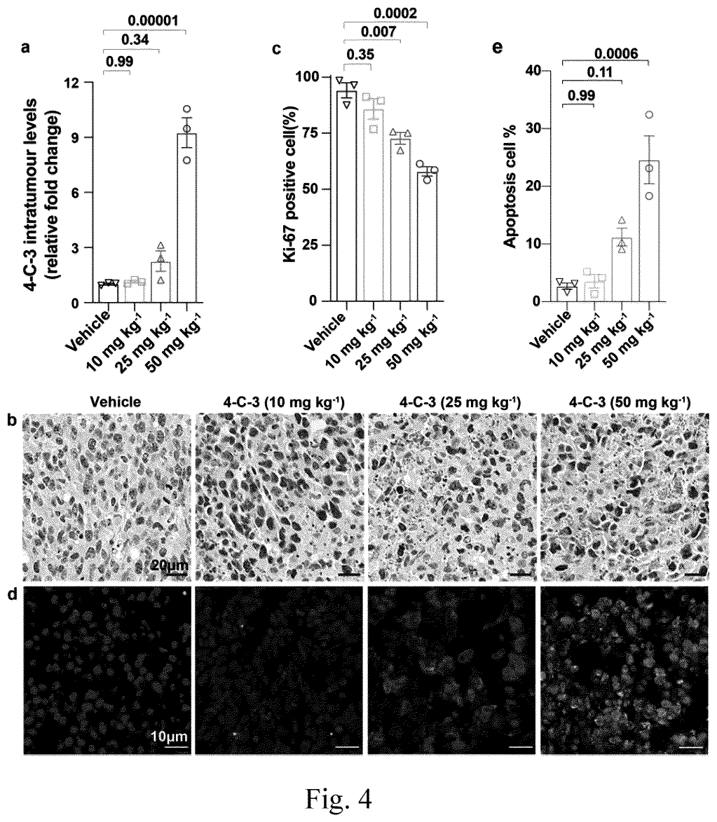

To confirm the clinical relevance of our findings obtained from the in vitro assays, we implanted DLD-1 and (FIG. 3a) and MC38 (FIG. 3e) colon cancer cells subcutaneously into nude mice to generate xenograft tumors. The mice were then gavaged daily with 4-C-3 for 16 days. Treatment with 4-C-3 significantly suppressed DLD-1 (FIG. 3b-d) and MC38 (FIG. 3f-h) tumor growth in a dose-dependent manner. We next verified that treatment with 4-C-3 suppressed the orthotopic tumor growth of the luciferase-labelled CRC cell line HCT116 (FIG. 3i). 4-C-3 significantly reduced tumor size (FIG. 3j-l), and intratumoral 4-C-3 levels were markedly increased (FIG. 4a). Consistently, a marked decrease in Ki67 levels (FIG. 4b-c) and an increase in TUNEL-positive cells (FIG. 4d-e) were observed in 4-C-3-treated tumors compared with vehicle-treated controls.

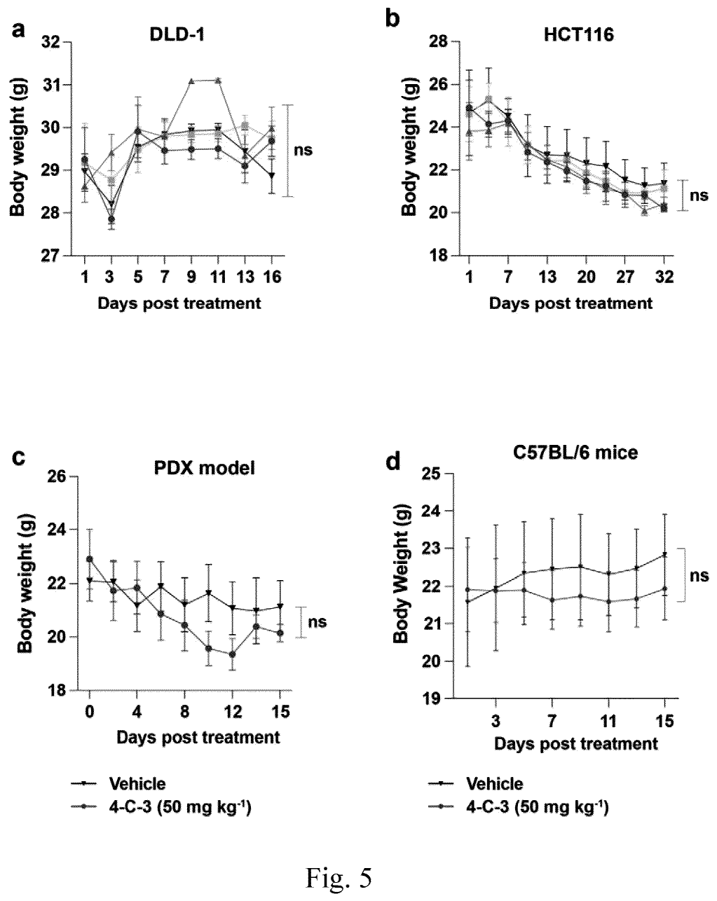

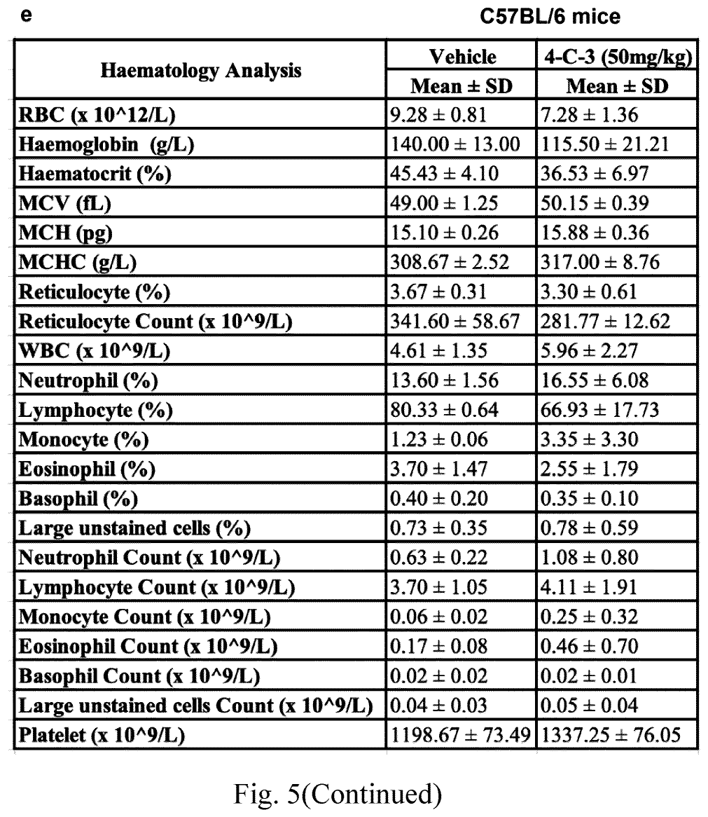

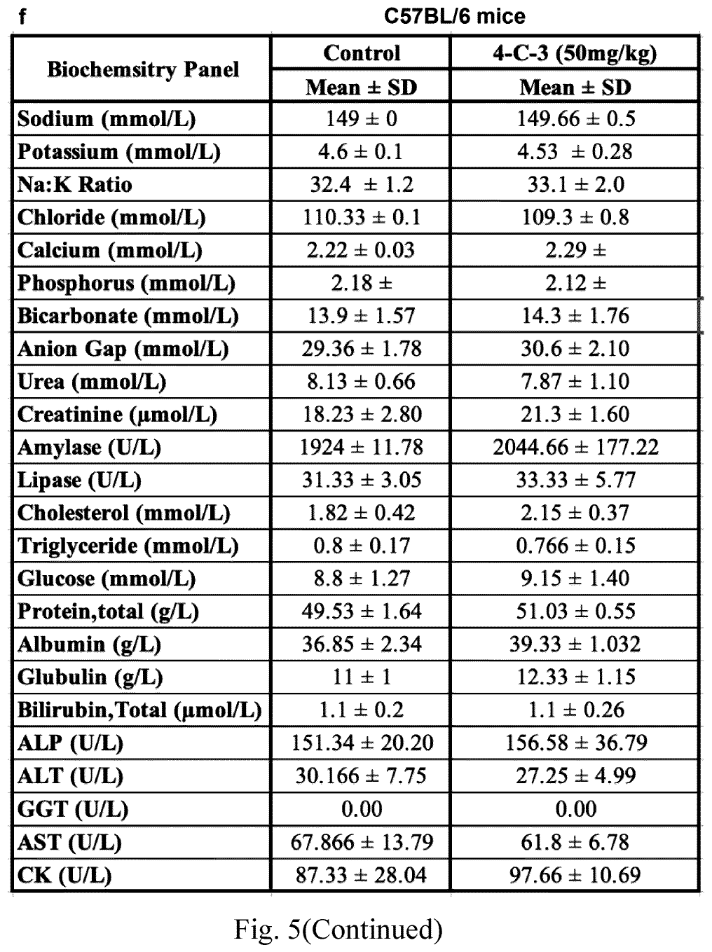

Notably, 4-C-3 treatment did not induce any adverse side effect as examined by body weight changes in CRC models (FIG. 5a-c). Furthermore 4-C-3 did not alter normal tissue homeostasis, as assessed by body weight (FIG. 5d) and regular blood tests (FIG. 5e-f). These in vitro and in vivo data, with the support of multiple tumor models, demonstrate a safe and potent effect of 4-C-3 in modulating CRC growth.

### 3. 4-C-3 Inhibits the Growth of CRC Cells by Antagonizing the EGFR Pathway

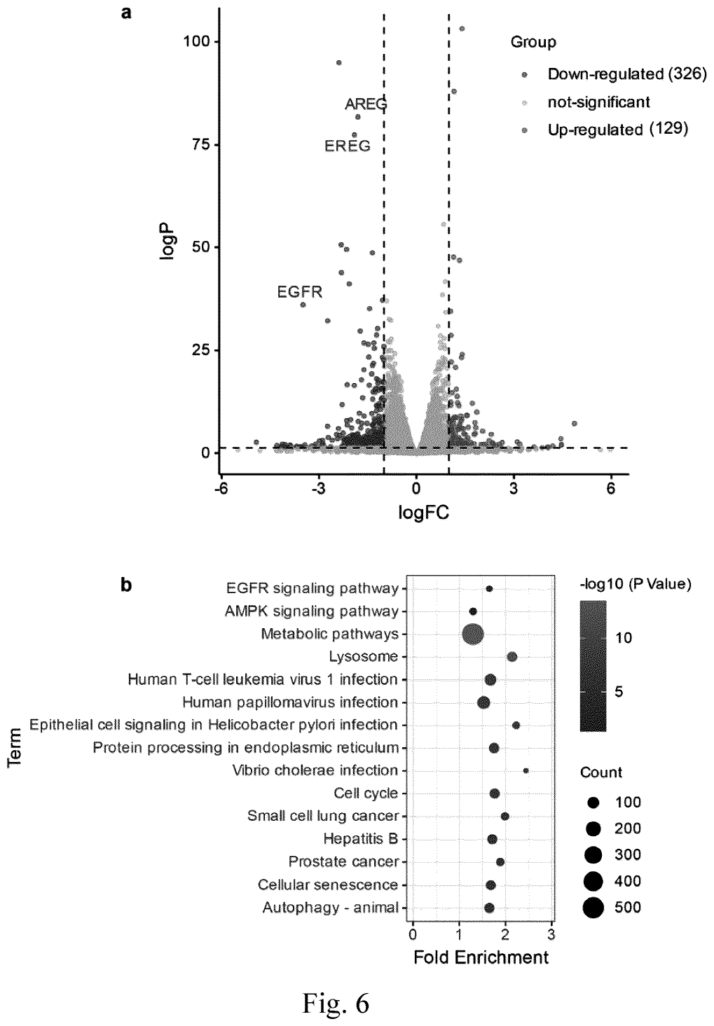

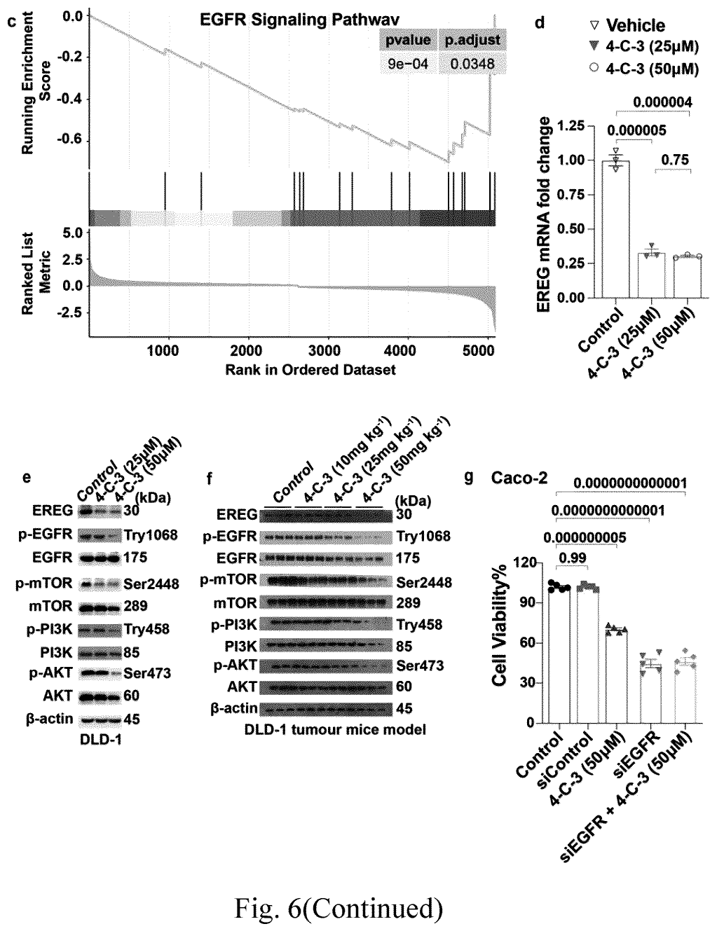

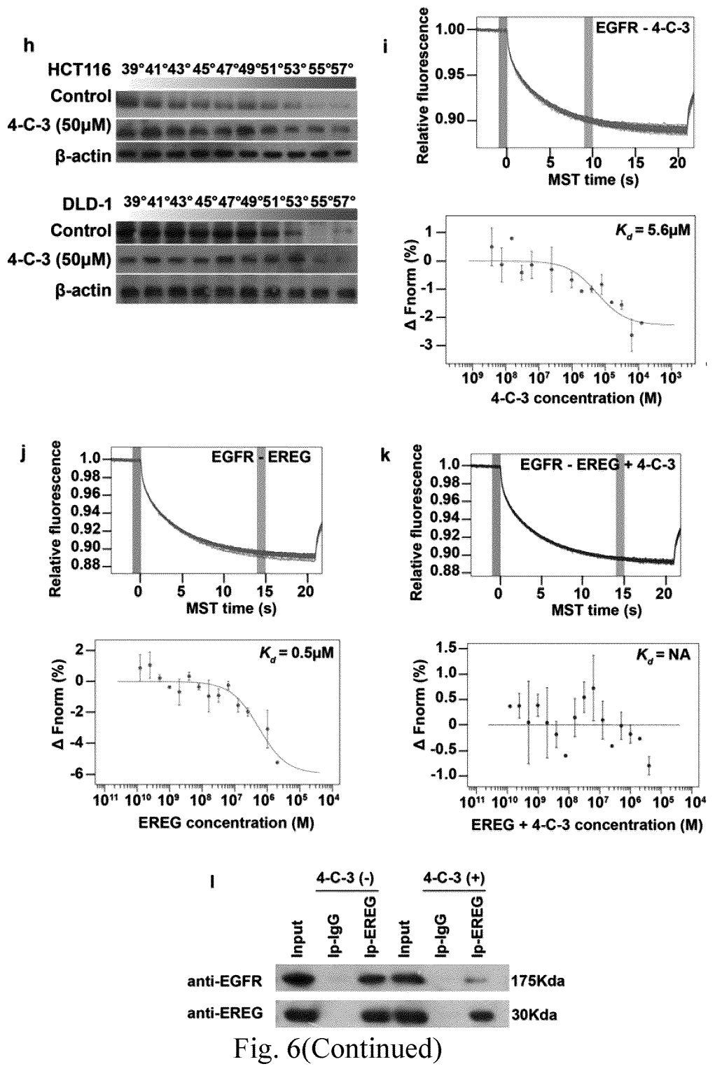

In our efforts to provide molecular and mechanistic insights into the anti-CRC effects of 4-C-3, we employed transcriptomics to determine the gene modules regulated by 4-C-3 in CRC cells. RNA sequencing studies in DLD-1 cells treated with 4-C-3 revealed dramatic transcriptional changes induced by 4-C-3 (FIG. 6a). Volcano plot analysis demonstrated that 129 and 326 genes were up-regulated and down-regulated by 4-C-3, respectively, as shown in FIG. 6a. KEGG pathway enrichment analysis of down-regulated genes identified the EGFR signaling pathway as the top enriched pathway (FIG. 6b). Gene set enrichment analysis (GSEA) confirmed suppressed EGFR signaling in 4-C-3-treated cells (FIG. 6c). Consistently, qPCR analyses validated that the expression of epiregulin (EREG), a ligand of EGFR that is highly expressed in CRC tumors and regulated by an autocrine loop through EGFR downstream signaling activation, was significantly reduced in DLD-1 cells treated with 4-C-3 (FIG. 6d). As a complementary approach, we observed that treatment with 4-C-3 significantly inhibited the phosphorylation of EGFR and its downstream signaling targets, including PI3K, Akt and m-TOR, in the DLD-1 cell line (FIG. 6e). A similar observation was obtained for orthotopic DLD-1 tumors from 4-C-3-treated mice (FIG. 6f). The inhibitory effect of 4-C-3 on the growth of Caco-2 cell line was abrogated by EGFR ablation with siRNA (FIG. 6g), confirming that EGFR signaling is an intermediary of the inhibitory effect of 4-C-3 on CRC growth.

To elucidate the mechanism of EGFR inhibition by 4-C-3, we investigated the potential interaction between EGFR and 4-C-3 via a cellular thermal shift assay in HCT116 and DLD-1 cell lines. In relative to the control, EGFR exhibited thermal shifts in the presence of 4-C-3 at the denaturation temperate ranging from 51° C. to 57° C., revealing the cell-level interaction between EGFR and 4-C-3 (FIG. 6h). We then performed microscale thermophoresis (MST) analyses on binding between the recombinant extracellular domain of EGFR (EGFR-ECD) and 4-C-3. EREG, one of the top downregulated targets by 4-C-3 in our transcriptome analyses (FIG. 6a), acted as a positive control for the assay. We established that 4-C-3 directly bound to the recombinant extracellular domain of EGFR with a K_{d} value of 5.6 μM while EREG had a K_{d} value of 0.5 μM (FIG. 6i-j). Although EREG bound EGFR-ECD with higher affinity, the binding of EREG was completely abrogated when EGFR-ECD was co-incubated with EREG and 4-C-3 (FIG. 6k). By performing immunoprecipitation with Caco2 cells, we confirmed that 4-C-3 efficiently inhibited the cell-surface binding of EREG to EGFR (FIG. 6l), revealing the ability of 4-C-3 to block the ligand binding to EGFR.

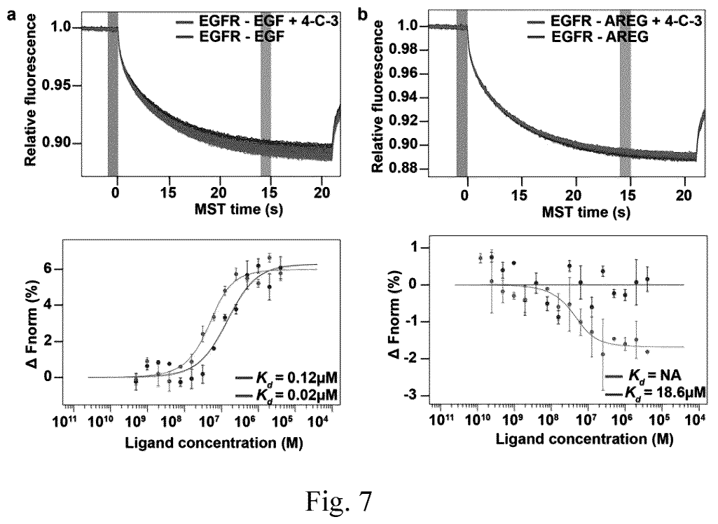

To further validate this observation, we performed additional MST experiments with a high affinity ligand EGF and a low affinity ligand AREG. 4-C-3 similarly suppressed the bindings of EGF and AREG to EGFR-ECD (FIG. 7a-b). Specifically, as shown in FIG. 7a, the dissociation constant (K_{d}) for EGFR-EGF binding increased from 0.02 μM to 0.12 μM upon addition of 4-C-3, indicating reduced binding affinity. Furthermore, as depicted in FIG. 7b, the binding of EGFR to AREG (K_{d}=18.6 μM) was completely abolished in the presence of 4-C-3, as evidenced by an undetectable K_{d} value.

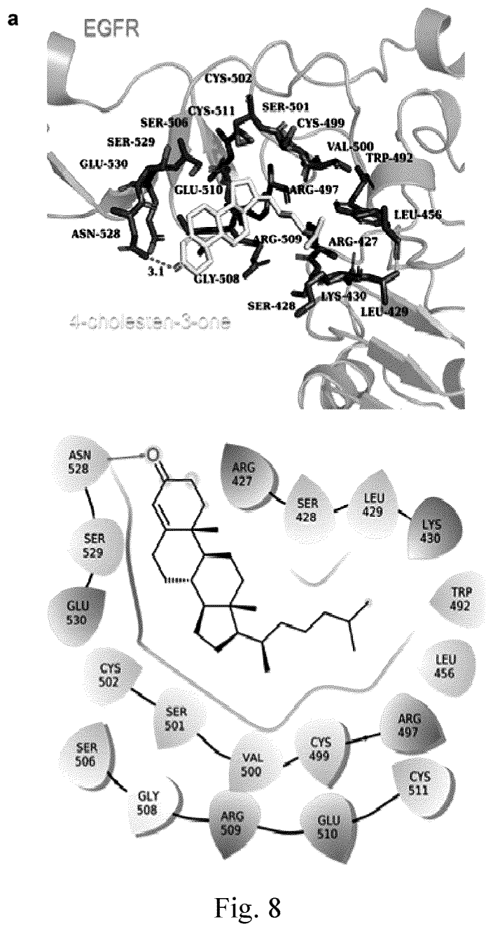

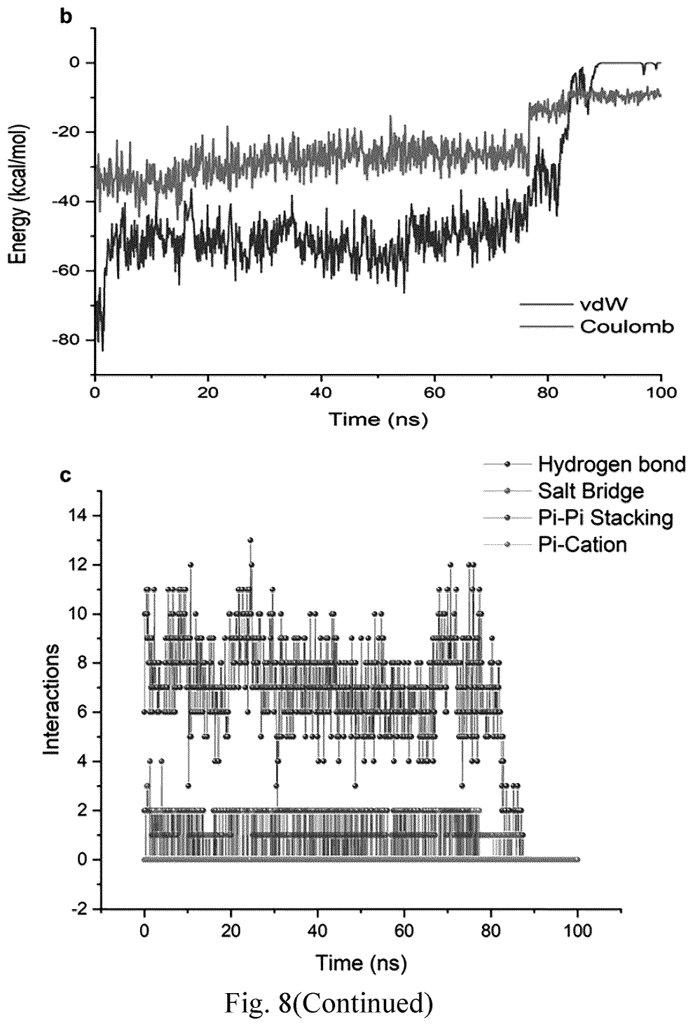

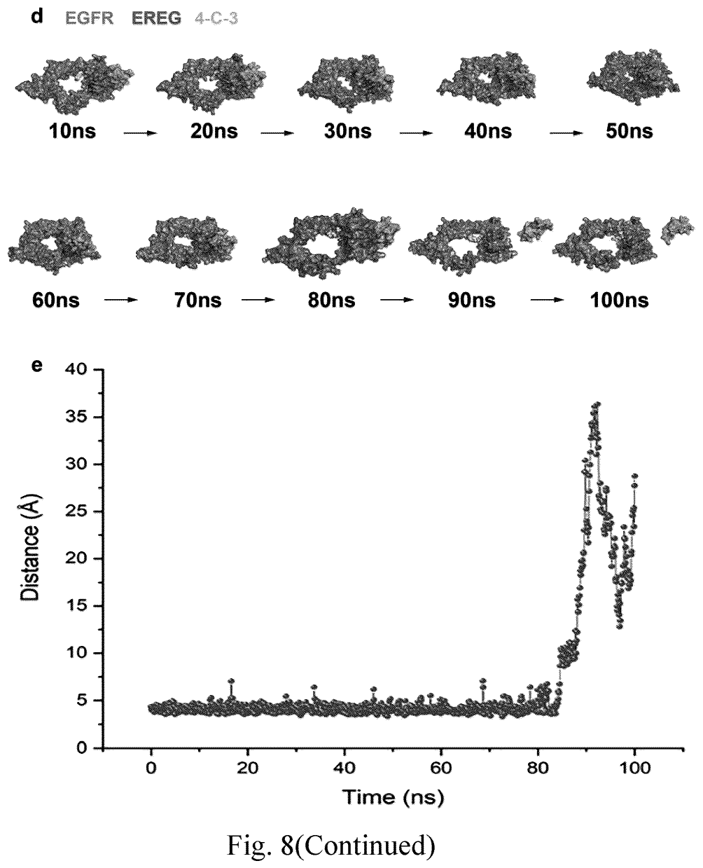

To determine the residues of EGFR involved in binding with 4-C-3, we docked 4-C-3 into an AlphaFold-modelled EGFR. The binding pocket of EGFR-ECD around 4-C-3 was calculated to be at a distance of 3.1 Å. In the modelled binding pocket, 4-C-3 interacts with Asn528 in the EGFR binding pocket via H-bond formation (FIG. 8a). To further understand how 4-C-3 interrupts ligand binding to EGFR, we executed 100 ns molecular dynamics (MD) simulations coupled with mechanics and thermodynamic calculations to study the dynamical structural characteristics and interactions between them (FIG. 8b-e). During the MD simulation, EREG was initially placed in the binding site of EGFR at a distance of 4 Å with a binding free energy (vdW=−75.39 kcal/mol; Coulomb=−31.02 kcal/mol) (FIG. 8b). This binding was stabilized by multiple non-bonded interactions, including hydrogen bonding, salt bridge interactions, π-π stacking interactions and π-cation interactions (FIG. 8c). Upon the binding of 4-C-3, EGFR underwent gradual conformational changes, leading to the destabilization of EREG/EGFR complex (FIG. 8d). By 90 ns, EREG was completely dissociated from EGFR as evidenced by the increased binding distance (36 Å), the reduced binding energy (vdW=0 kcal/mol; Coulomb=−8 kcal/mol) and the disruption of all non-bonded interactions (FIG. 8e). Taken together, these findings reveal that 4-C-3 is a potent endogenous inhibitor of EGFR and that it inhibits the growth of CRC cells by antagonizing the EGFR pathway.

### 4. 4-C-3 Attenuates Resistance to Common Anti-EGFR Therapies in CRC

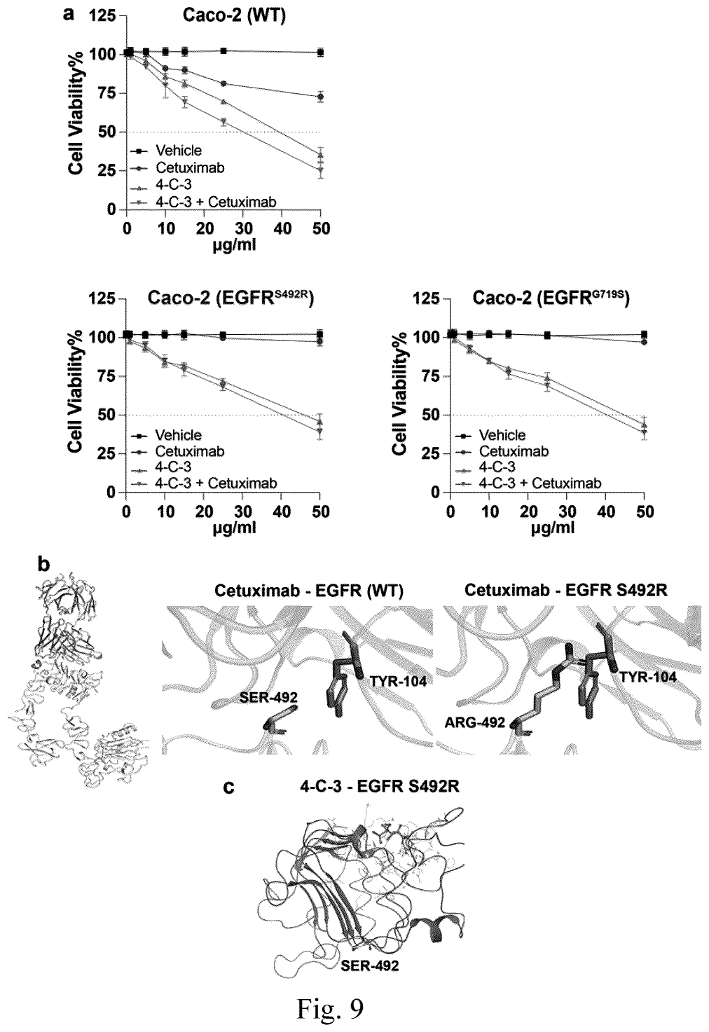

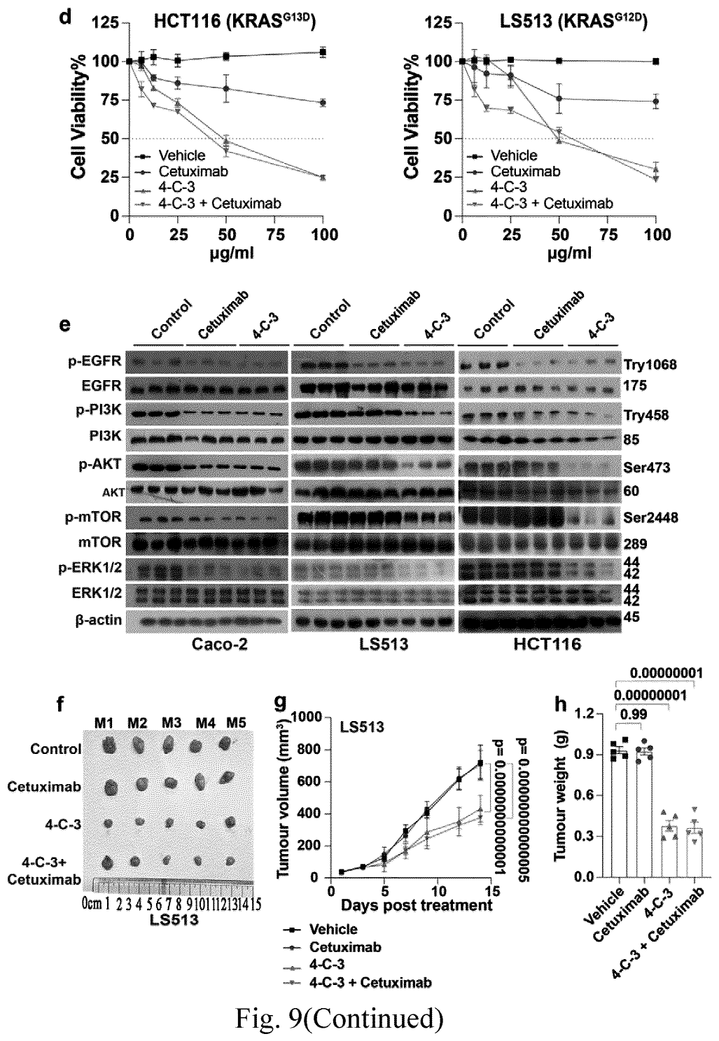

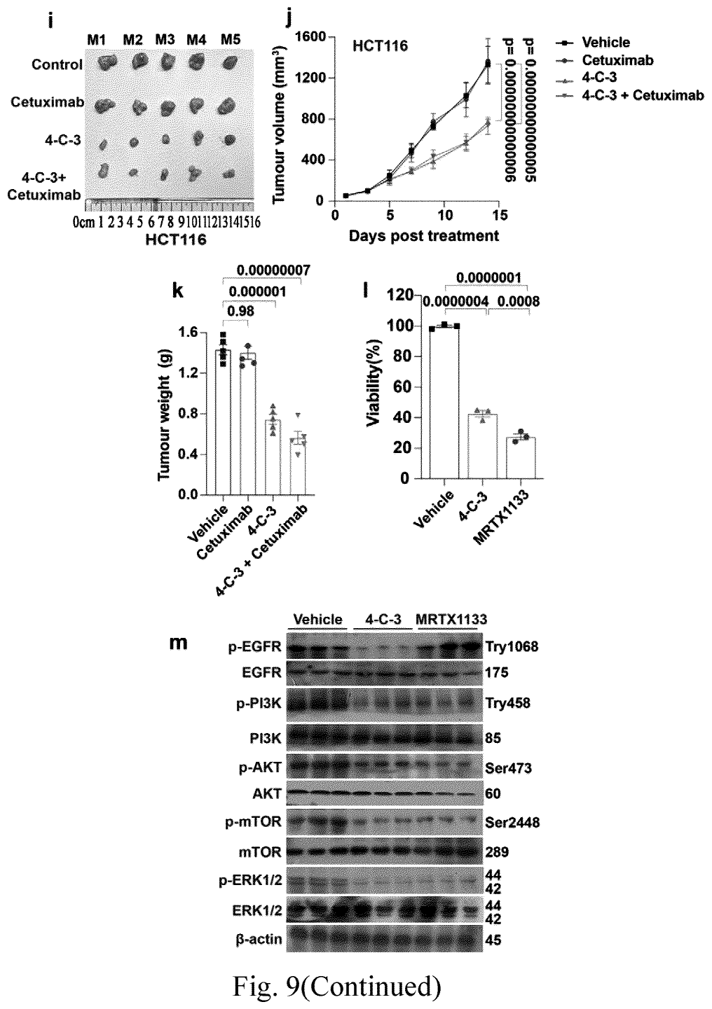

Anti-EGFR therapies, such as cetuximab and panitumumab, are effective therapeutic agents for metastatic CRC. However, resistance to targeted therapies limits their clinical use and efficiency. To investigate whether 4-C-3 delivery can attenuate resistance to common anti-EGFR therapies in CRC, the inhibitory effect of 4-C-3 on CRC growth was examined in Caco-2 cells expressing wild-type EGFR or mutant EGFRs with common drug resistance mutations (EGFR^{S492R} and EGFR^{G719S}). Treatment with cetuximab served as a control. Unlike Caco-2 cells with wild-type EGFR, cells expressing mutant EGFRs were all resistant to cetuximab at our tested dosages (FIG. 9a). In contrast, 4-C-3 at the same molar dose effectively suppressed the growth of these EGFR-mutant cells (FIG. 9a), suggesting that 4-C-3 attenuates resistance to common EGFR resistance mutations. Furthermore, molecular docking analysis showed that 4-C-3 interacts with an epitope on EGFR that is distinct from the cetuximab epitope (FIG. 9b-c), revealing the potential mechanism by which 4-C-3 overcomes cetuximab resistance in CRC.

KRAS mutations are present in approximately 40% of patients with colorectal cancer. In colorectal cancer, the presence of KRAS mutations is a significant predictor of resistance to epidermal growth factor receptor (EGFR) inhibitors. These mutations, often found in codons 12 and 13 of the KRAS gene, lead to constitutive activation of the RAS/RAF/MEK/ERK signaling pathway, rendering EGFR-targeted therapies ineffective. In contrast, agents targeting KRAS mutations have limited efficacy against KRAS-mutant CRC in part because of EGFR-mediated reactivation of MAPK signaling, prompting clinical trials with combined therapies targeting KRAS mutations and EGFR in CRC. However, secondary resistance mechanisms driven by the compensatory activation of other tyrosine kinases or the upregulation of mTOR signaling limit the response to the combination strategy. Given the EGFR-PI3K-mTOR-inhibitory profile of 4-C-3, we postulated that 4-C-3 may be efficacious against KRAS-mutant CRC. We indeed found that 4-C-3 also had potent inhibitory effects on the proliferation of KRAS-mutant CRC cell lines, including HCT116 (KRAS^{G13D}) and LS513 (KRAS^{G12D}), both of which were resistant to cetuximab (FIG. 9d). While cetuximab was able to decrease EGFR activation in LS513 and HCT116 cells, the cells maintained a high level of PI3K/mTOR signaling activation that was not suppressed by drug treatment. In contrast, 4-C-3 effectively inactivated the phosphorylation of EGFR and its downstream signaling targets (m-TOR, PI3K, AKT and Erk1/2) in these cell lines (FIG. 9e). To validate the anti-tumor efficacy of 4-C-3 on KRAS mutant CRC in vivo, we treated nude mice bearing subcutaneous LS513 or HCT116 xenografts with either cetuximab or 4-C-3. As expected, 4-C-3 conferred potent inhibitory effects on the growth of LS513 and HCT116 xenografts, whereas cetuximab had limited effects (FIG. 9f-k).

In addition, we further tested the anti-tumor effect of 4-C-3 on CRC organoids derived from Apc^{min/+} In Kras^{G12D/+} mutant mice. Treatment with 4-C-3 suppressed the growth of mutant KRAS-CRC organoids, with potency comparable to that of the KRAS^{G12D} inhibitor MRTX1133 (FIG. 91). Consistently, western blot analysis showed that 4-C-3 inhibited EGFR-MAPK signaling activation in these organoids (FIG. 9m). These findings demonstrate that 4-C-3 holds promise for overcoming mutant KRAS-driven resistance to anti-EGFR therapies in the treatment of CRC.

### 5. 4-C-3 is a Non-Covalent Small Molecule Inhibitor of KRAS Oncogenic Variants

Our observations that 4-C-3 is efficacious against multiple KRAS-mutant CRC cells are surprising, given the expectation that KRAS oncogenic mutations are causally implicated in resistance to EGFR blockage in CRC, and the inhibitory effect of 4-C-3 on EGFR activation is theoretically not sufficient to attenuate the growth of KRAS-mutant CRC cells. This prompted us to explore additional mechanisms that may contribute to the ability of 4-C-3 to target KRAS-mutant CRC cells.

In this study, we focused on the KRAS p.G12D for investigation, as the KRAS^{G12D} mutation is the most common KRAS mutation in CRC, and there is currently no FDA-approved drug for targeting cancers with this mutation. Using MST assays, we found that 4-C-3 exhibited 3-fold selectivity for binding to GDP-bound KRAS^{G12D} compared with GDP-bound KRAS^{WT} (GDP-bound KRAS^{G12D}) K_{d}=7.2 μM versus GDP-bound KRAS^{WT} K_{d}=21.3 μM) (FIG. 10a). To examine the potential effects of 4-C-3 on KRAS functions, fluorescent-based biochemical assays were employed to study the rate of GDP/GTP exchange reactions of KRAS. 4-C-3 reduced the rates of both intrinsic and SOS1-mediated nucleotide exchange reactions for KRAS^{G12D}, with IC₅₀ values of 14.32 μM and 12.57 μM, respectively (FIG. 10b-c). For KRAS^{WT}, 4-C-3 had weaker effects, with IC₅₀ values of 30.79 μM and 28.4 μM, respectively (FIG. 10b-c). This indicates that 4-C-3 exerts stronger inhibition on KRAS^{G12D} than on KRAS^{WT}.

To investigate the effect of 4-C-3 on effector binding, we tested the potency of 4-C-3 to displace the RAS-binding domain of CRAF from purified KRAS variants preloaded with GMPPNP, a non-hydrolysable GTP analog. 4-C-3 diminished the active KRAS^{G12D}-CRAF interaction with an IC₅₀ value of 41.14 μM, but did not affect the interaction between CRAF and KRAS^{WT} at this concentration (FIG. 10d), which again suggests that 4-C-3 is more selective for KRAS^{G12D}. To further confirm the interaction between 4-C-3 and KRAS at the cellular level, we performed traditional cellular thermal shift assays in Caco-2 (KRAS^{WT}) and LS513 (KRAS^{G12D}) cell lines. Compared to the control, KRAS exhibited significant thermal shifts in the presence of 4-C-3 in LS513 cells, whereas this thermal shift was less prominent in Caco-2 cells (FIG. 10e), revealing the intracellular interaction between KRAS and 4-C-3 and the selectivity of 4-C-3 towards KRAS^{G12D} binding. To decipher the regulatory role of 4-C-3 in KRAS signaling, we performed an active RAS (RAS-GDP) pull-down assay in the cell lysate of LS513 cells treated with 4-C-3. Our results showed that 4-C-3 reduced the level of KRAS-GTP in LS513 cells (FIG. 10f). Additionally, 4-C-3 effectively suppressed the phosphorylation of Erk1/2, a key downstream pathway in KRAS signaling, in these cells, whereas cetuximab had limited effects (FIG. 9e).

To better understand how 4-C-3 interacts with KRAS, we docked 4-C-3 into an AlphaFold-modelled GDP-bound KRAS^{G12D}. 4-C-3 was predicted to bind in the switch-II pocket of KRAS though H-bond formation with Asp12, an interaction that could not be observed in KRAS^{WT} (FIG. 10g). Furthermore, 4-C-3 demonstrates enhanced binding affinity towards various KRAS mutants (FIG. 10h) and effectively inhibits the activation of multiple KRAS variants, including G12C, G13C, and G13D, with potencies comparable to that observed for the G12D mutant (FIG. 10i). These findings indicate that 4-C-3 possesses broad inhibitory activity across diverse KRAS mutant forms. We next performed long timescale simulations to reveal the interaction dynamics of the 4-C-3-KRAS^{G12D} complex (FIG. 11a-d). The protein backbone root-mean-square fluctuation (RMSF) values showed that the KRAS^{G12D}-GDP-4-C-3 system reached equilibrium at 8 ns, while the KRAS^{G12D}-GDP system in the absence of 4-C-3 did not reach equilibrium within 30 ns (FIG. 11a-b). This indicates that 4-C-3 stabilizes the KRAS^{G12D}-GDP complex. In contrast, the RMSD values for the KRAS^{WT}-GDP-4-C-3 and KRAS^{WT}-GDP systems showed that equilibrium was reached at 12 ns with 4-C-3 and at 16 ns without it (FIG. 11c-d). This suggests that 4-C-3 only slightly stabilizes the KRAS^{WT}-GDP complex. To validate these simulations, we performed MST assays on the binding of GDP on KRAS^{G12D}. Upon the addition of 4-C-3, the binding between GDP and KRAS^{G12D} increased by approximately 6-fold (FIG. 11e-f). These results suggest that the binding of 4-C-3 locks KRAS^{G12D} in an inactive, GDP-bound state. These results collectively suggest that the dual-targeting of EGFR and KRAS with 4-C-3 is an effective therapeutic strategy for overcoming mutant KRAS-driven resistance to anti-EGFR therapies in CRC.

### 6. The Development of Functional Engineered Commensal Microbes for the Treatment of CRC

In solid tumors, especially CRC, high levels of cholesterol are often observed; this phenomenon has been linked to increased tumor proliferation and progression. Given the potent inhibitory effect of the cholesterol metabolite 4-C-3 on CRC growth, we next investigated whether microbial catabolism of cholesterol into 4-C-3 by the gut microbiota can inhibit CRC tumorigenesis. To facilitate the conversion of cholesterol to 4-C-3 in the tumor, we transformed a series of nonpathogenic strains of E. coli including E. coli. (TOP 10), E. coli. (JM109) and E. coli. (Nissle 1917) with a single plasmid that encodes cholesterol oxidase (CHOX) from marine Streptomyces sp., an enzyme that can specifically catalyze the oxidation and isomerization of cholesterol to 4-C-3, under the control of the constitutive promoter J23119 (P19) (FIG. 12a). In in vitro culture with a medium containing cholesterol, we found that only the engineered E. coli. (TOP 10) bacteria expressing CHOX could efficiently convert cholesterol to 4-C-3, as assessed by liquid chromatography-tandem mass spectrometry (LC-MS) analysis (FIG. 12b-c). This 4-C-3-producing strain is hereafter referred to as E. coli CHOX⁺ bacteria. The optimized gene sequence of CHOX is described in FIG. 12d.

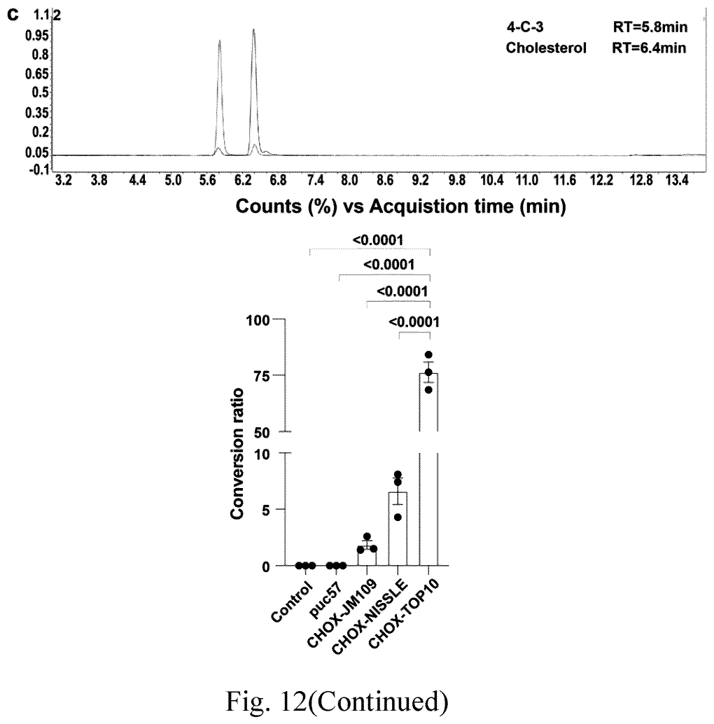

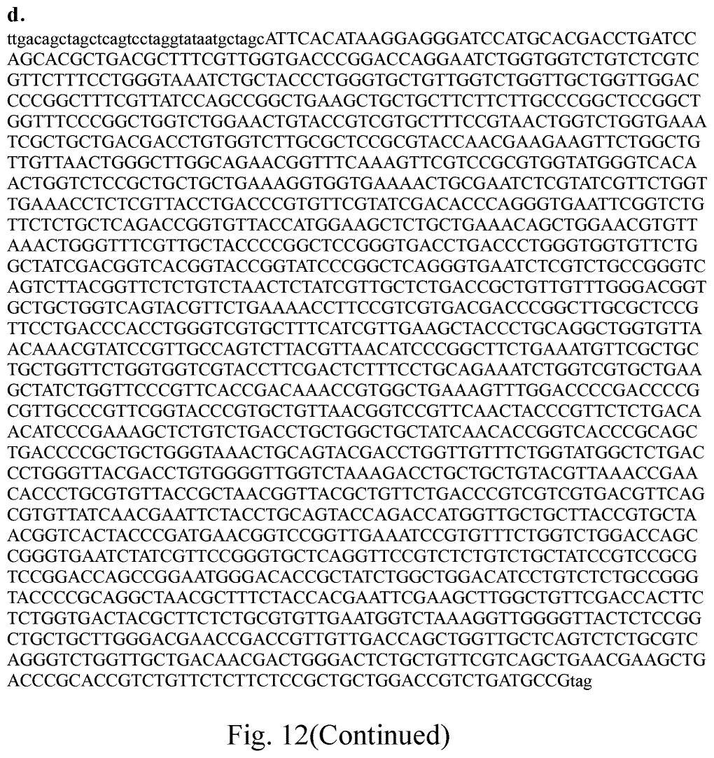

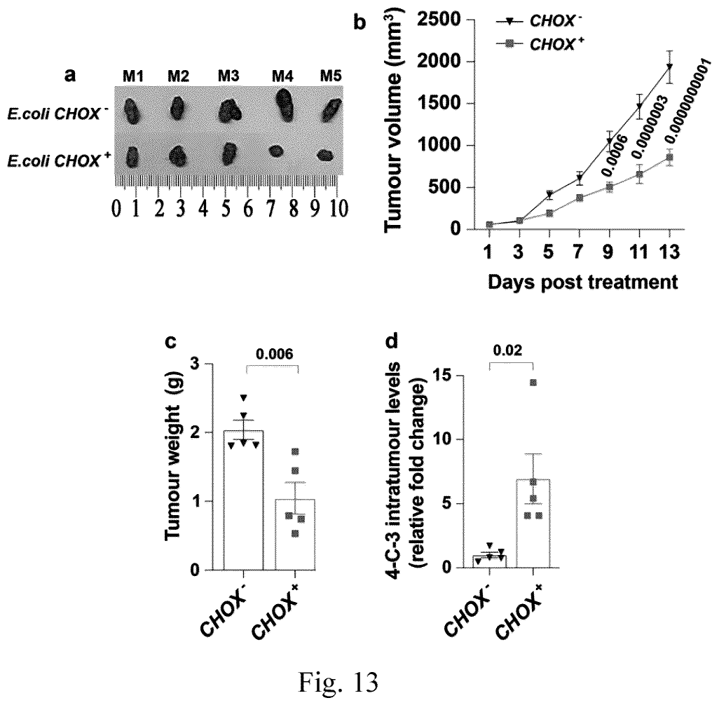

To study the in vivo capacity of engineered E. coli CHOX⁺ bacteria in producing 4-C-3, we subcutaneously implanted MC38 colon cancer cells into nude mice to generate xenograft tumors. The mice were then gavaged daily with E. coli CHOX⁺ bacteria or E. coli CHOX⁻ bacteria. The colonization of E. coli CHOX⁺ bacteria but not E. coli CHOX⁻ bacteria significantly reduced MC38 tumor growth after treatment initiation (FIG. 13a-c). The level of 4-C-3 was significantly increased in treated tumors (FIG. 13d), confirming the successful delivery of 4-C-3 to the tumor by our engineered bacteria.

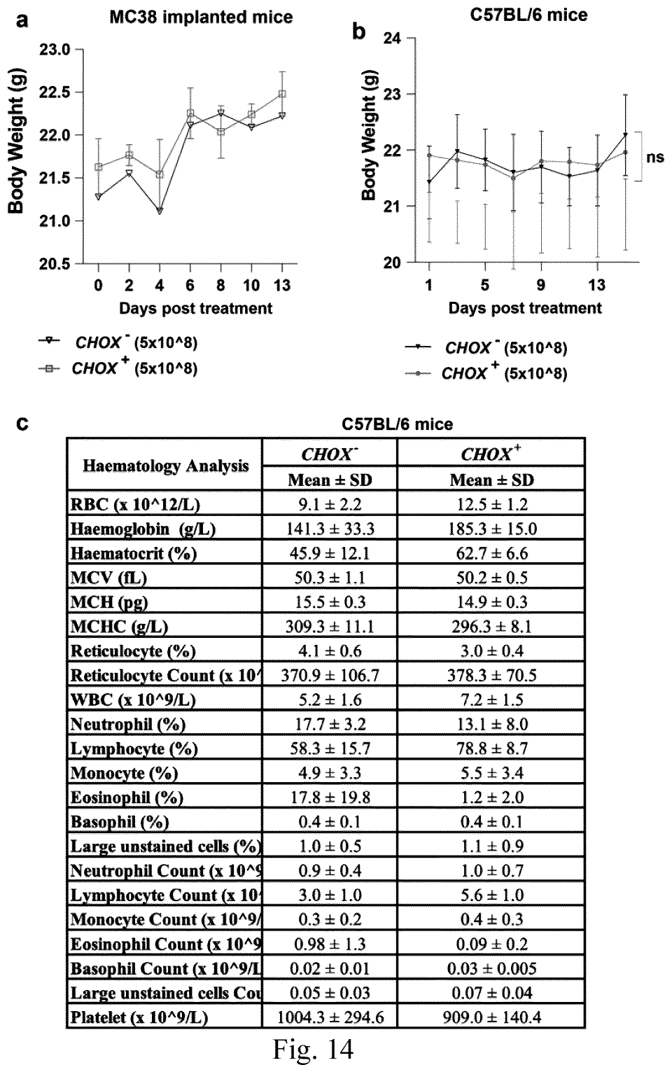

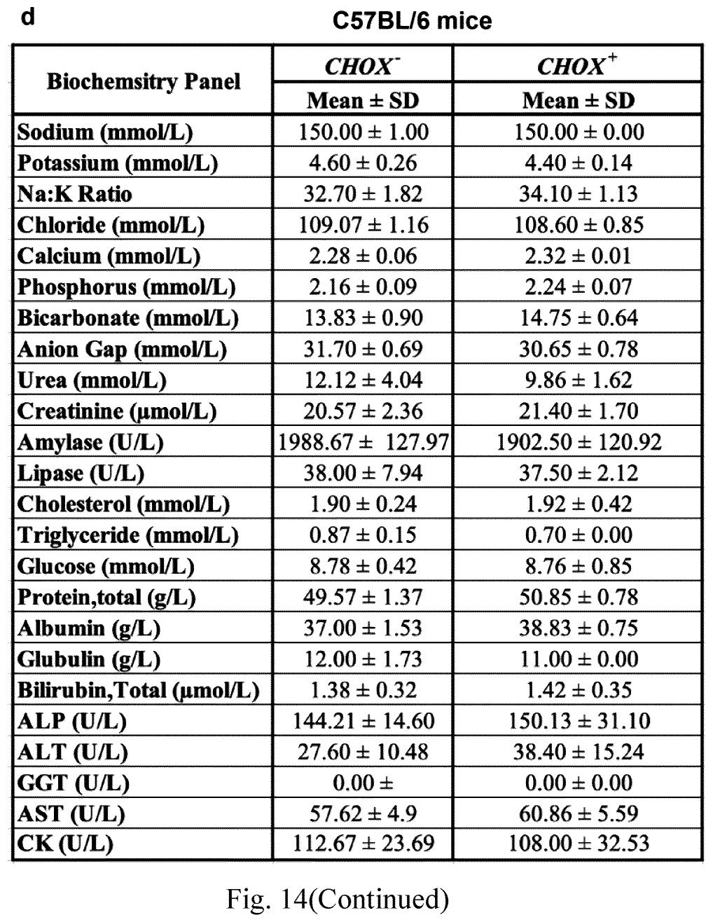

Notably, the colonization of E. coli CHOX⁺ bacteria exhibited an excellent safety profile in this cancer model, as assessed by body weight (FIG. 14a). Furthermore E. coli CHOX⁺ bacteria showed no adverse effects in healthy mice as shown by body weight and regular blood tests results (FIG. 14b-d).

### 7. Oscillibacter Ruminantium-Mediated Cholesterol Metabolism Protects Against CRC Tumorigenesis

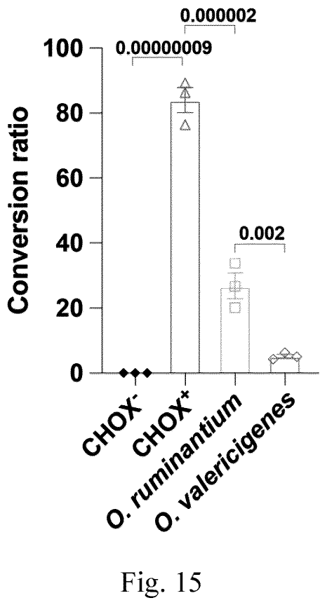

Some Oscillibacter species, culturable gut bacteria with relatively high levels in the human gut microbiome, can metabolize cholesterol and produce its metabolites, including 4-C-3. To investigate whether these bacterial species are responsible for cholesterol dysregulation in CRC, we analyzed our previously published datasets obtained from metagenomic analyses in our CRC patient cohort (Kong et al., 2023; Li et al., 2024b; Yang et al., 2021) and identified that O. ruminantium could convert cholesterol into 4-C-3 in in vitro culture (FIG. 15). Unlike E. coli CHOX⁺ bacteria, which specifically convert cholesterol into 4-C-3, O). ruminantium produces both 4-C-3 and its downstream metabolites. This explains the higher capacity of E. coli CHOX⁺ bacteria in producing 4-C-3 (FIG. 15). The abundance of O. ruminantium was reduced in CRC patients when compared with healthy controls (FIG. 16a).

To investigate whether the conversion of cholesterol into 4-C-3 by O. ruminantium similarly exerts anti-tumor effects on CRC as observed with E. coli CHOX⁺ bacteria, we daily gavaged mice bearing orthotopic HCT116 xenograft tumors with O. ruminantium (DSM113516), E. coli CHOX⁺ bacteria or 4-C-3 for 21 days. Oscillibacter valericigenes (O. valericigenes), which could not produce 4-C-3, served as a control. All treatment groups, except for the colonization with O. valericigenes, significantly suppressed tumor growth, with the strongest effects seen in mice colonized with E. coli CHOX⁺ bacteria (FIG. 16b-d), which correlated with the higher capacity of E. coli CHOX⁺ bacteria to convert cholesterol into 4-C-3 (FIG. 16e-f). Consistently, similar results were obtained in a murine model of CRC induced with a combination of the carcinogen azoxymethane (AOM) and colitis-inducing dextran sodium sulfate salt (DSS) over a period of 14 weeks (FIG. 16g). All treatment groups, except for O. valericigenes, increased colon length, and reduced tumor load and number, with the most robust effect observed in the colonization of E. coli CHOX⁺ bacteria (FIG. 16h-j). Along with the reduction in intratumoral cholesterol (FIG. 16j), the level of 4-C-3 increased significantly in the tumors treated with O. ruminantium or E. coli CHOX⁺ bacteria (FIG. 16k).

Next, we investigated the ability of O. ruminantium to mitigate mutant KRAS-induced CRC tumorigenesis. CRC organoids from Apc^{min/+}Kras^{G12D/+} mutant mice were subcutaneously implanted in immunocompetent mice. The mice were subjected to a 10-day regimen with either daily intratumoral injection of O. ruminantium (DSM113516) or E. coli CHOX⁺ bacteria, or oral administration of 4-C-3 or the KRAS^{G12D} inhibitor MRTX1133. Again, all treatment groups provoked anti-tumor effects on Kras^{G12D} CRC tumors, with the efficacy of E. coli CHOX⁺ bacteria comparable to that of MRTX1133 (FIG. 16l-p). Collectively, these data suggest that microbiome-mediated shifting of the balance from protumor cholesterol to antitumor 4-C-3 in the intratumoral environment may be an applicable therapeutic approach for the treatment of CRC.

8. Anti-Tumor Activity of 4-C-3 and Engineered E. coli CHOX⁺ Bacteria in Human Lung, Breast and Liver Cancer Models.

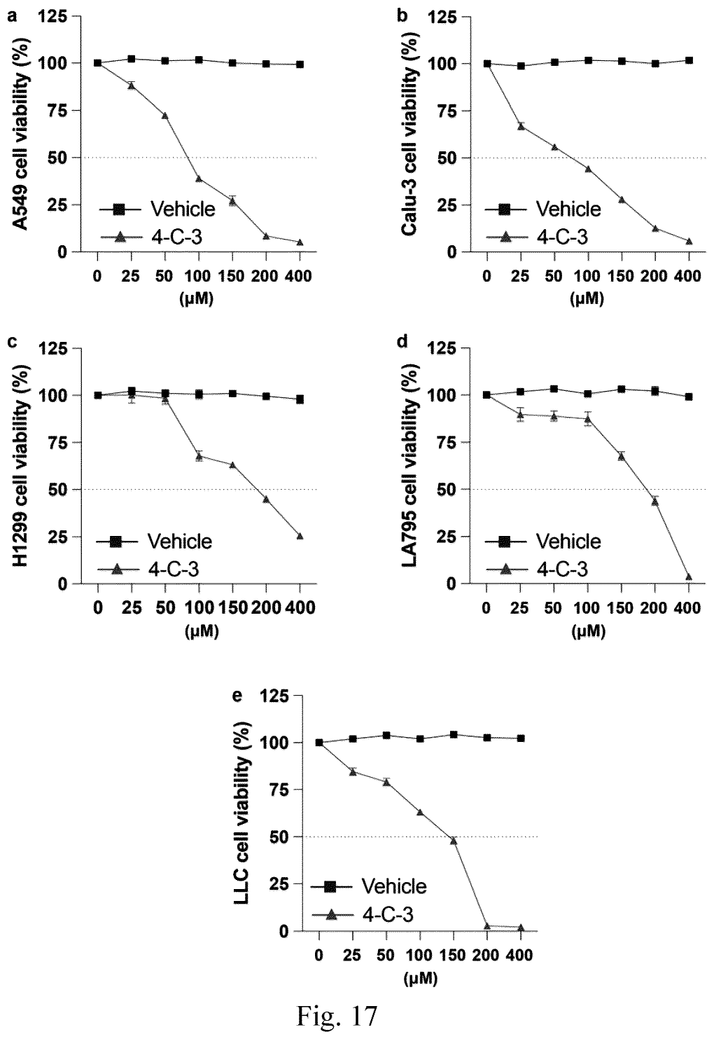

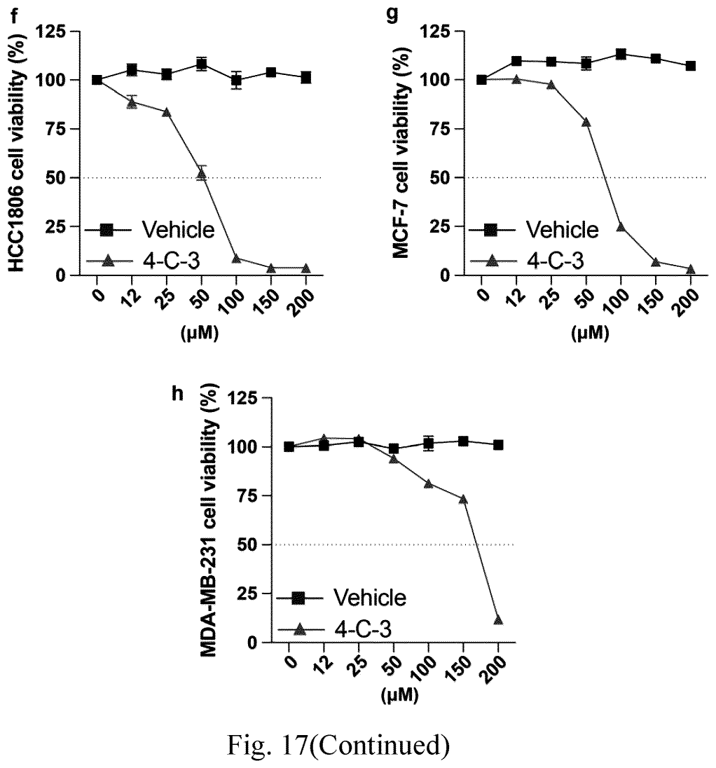

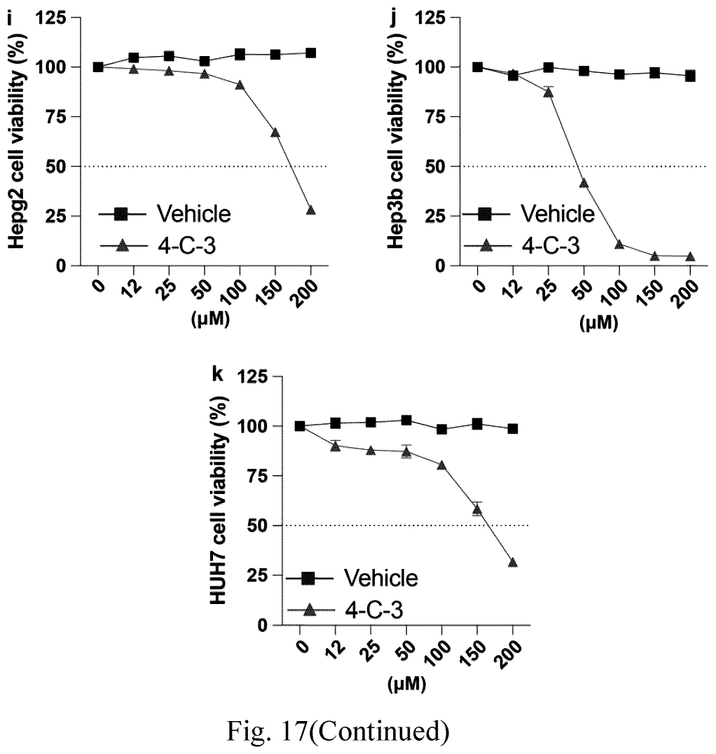

To investigate the anti-tumor activity of 4-C-3 in other cancer cells, MTT assay was conducted in a panel of human and mice lung cancer cell lines (A549, Calu-3, H1299, LA795, and LLC) (FIG. 17a-e), human breast cancer cell lines (HCC1806, MCF-7, MDA-MB-231) (FIG. 17f-h), and human liver cancer cell lines (Hepg2, Hep3b, HUH7) (FIG. 17i-k) treated with 4-C-3 at indicated dosages for 48 h. As shown in FIG. 17, 4-C-3 exhibited significant inhibitory effects on the growth of these cell lines as compared with the vehicle (0.1% ethanol), suggesting its efficacy against lung cancers.

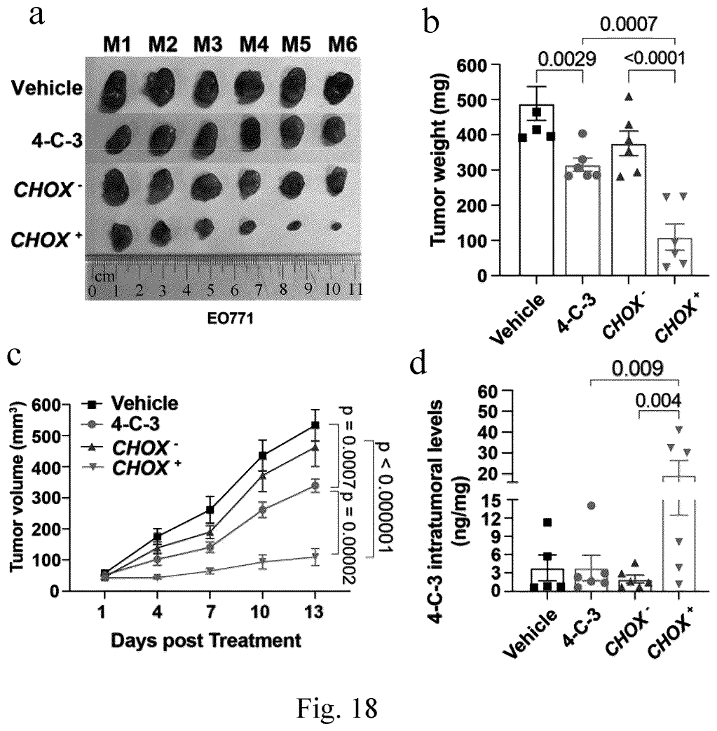

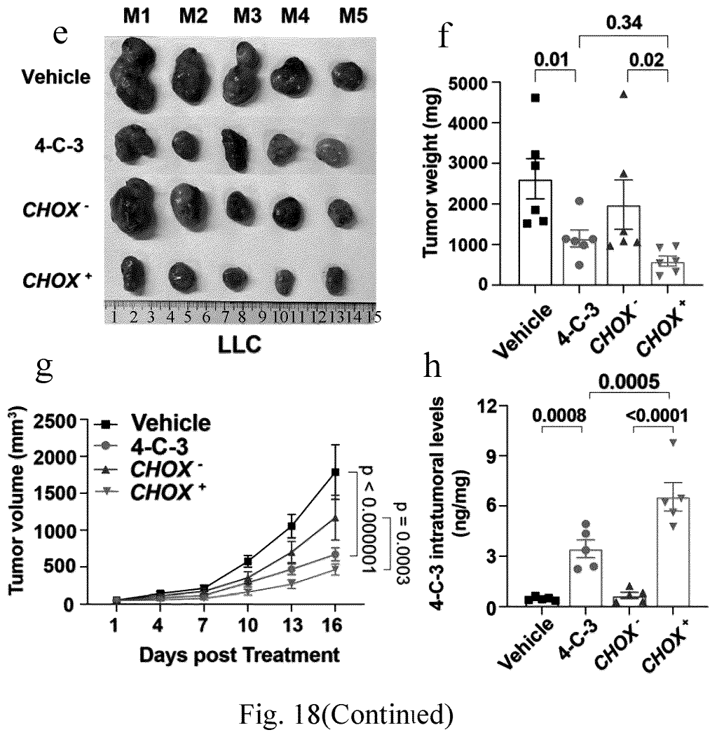

Furthermore, to investigate the in vivo therapeutic efficacy of engineered E. coli CHOX⁺ bacteria in additional cancer models, C57BL/6 mice were subcutaneously inoculated with either EO771 cells (for the breast cancer model) or LLC cells (for the lung cancer model) into both flanks. Following treatment initiation, intratumoral administration of E. coli CHOX⁺ bacteria, but not E. coli CHOX⁻ bacteria, resulted in a significant reduction in tumor growth (FIG. 18a-c, e-h). Moreover, levels of 4-C-3 were markedly increased in tumors treated with CHOX⁺ bacteria (FIG. 18d, h), confirming both the successful conversion of 4-C-3 to the tumor microenvironment by the engineered bacteria and the therapeutic effect in different cancer models.

What is claimed is:

1. A method of treating a cancer in a subject in need thereof, the method comprising administering to the subject a therapeutically effective amount of (i) a bacterium that converts intestinal cholesterol to 4-cholesten-3-one (4-C-3) or (ii) 4-C-3.

2. The method of claim 1, wherein the bacterium is selected from the group consisting of Oscillibacter species expressing cholesterol oxidase (CHOX), Eubacterium coprostanoligenes, a recombinant bacterium comprising a nucleic acid encoding CHOX, and any combination thereof, and wherein the recombinant bacterium is selected from the group consisting of bacteria of the genus Escherichia and genus Bacillus.

3. The method of claim 2, wherein the Oscillibacter species is Oscillibacter ruminantium and the recombinant bacterium is selected from the group consisting of an Escherichia coli bacterium, a Bacillus subtilis bacterium, and any combination thereof.

4. The method of claim 3, wherein the recombinant bacterium is selected from the group consisting of E. coli TOP10, E. coli DH5α, E. coli BL21, B. subtilis 168, and any combination thereof.

5. The method of claim 4, wherein the CHOX is selected from the group consisting of CHOX originating from Streptomyces species, such as Streptomyces fradiae, Streptomyces lavendulae, Streptomyces cavourensis, or any combination thereof.

6. The method of claim 1, wherein the 4-C-3 or the bacterium is administered in a therapeutically effective amount to achieve one or more or all of the following:
(i) reduce cholesterol concentrations within tumor tissues, gastrointestinal tract, serum, or feces of the subject;
(ii) increase the 4-C-3 concentrations within tumor tissues, gastrointestinal tract, serum, or feces of the subject;
(iii) inhibit the growth or viability of cancer cells;
(iv) reduce the size, weight, or volume of cancer tumors;
(v) promote apoptosis of cancer cells;
(vi) antagonize the EGFR pathway; and
(vii) target KRAS mutants.

7. The method of claim 1, wherein a fecal sample, a tumor sample, a plasm sample, or a serum sample obtained from the subject prior to administering a therapeutically effective amount of the bacterium or 4-C-3 exhibits at least one of: an increased cholesterol concentration and a decreased 4-C-3 concentration.

8. The method of claim 4 further comprising providing a sample obtained from the subject, wherein the sample is a fecal sample, a tumor sample, a plasma sample, or a serum sample; determining the concentration of at least one of 4-C-3 and cholesterol in the sample; and administering the 4-C-3 or the bacterium to the subject if a decreased concentration of 4-C-3 or an increased concentration of cholesterol is detected in the sample relative to an average concentration detected in healthy subjects.

9. The method of claim 8 further comprising co-administering one or more additional agents selected from the group consisting of: 5-cholesten-3-one, coprostanone, statins, chemotherapeutic agents, radiation therapeutic agents, immunotherapeutic agents, anti-EGFR therapeutic agents, agents targeting KRAS mutations, and any combination thereof.

10. The method of claim 9, further comprising co-administering anti-EGFR therapeutic agents, wherein the anti-EGFR therapeutic agent is selected from the group consisting of cetuximab, panitumumab, necitumumab, erlotinib, gefitinib, osimertinib, afatinib, dacomitinib, lapatinib, and any combination thereof.

11. The method of claim 1, wherein the cancer is selected from the group consisting of a gastrointestinal cancer, a lung cancer, a breast cancer, and any combination thereof.

12. The method of claim 11, wherein the gastrointestinal cancer comprises a gastrointestinal tract cancer selected from the group consisting of esophageal cancer, esophageal adenocarcinoma, gastric cancer, gastric adenocarcinoma, small intestine cancer, gastrointestinal lymphoma, colon cancer, rectal cancer, colorectal cancer, colorectal adenocarcinoma, anal cancer, appendiceal cancer, pancreatic cancer, pancreatic adenocarcinoma, and any combination thereof; a biliary tract cancer selected from the group consisting of a gallbladder cancer, a cholangiocarcinoma, and any combination thereof; a liver cancer; or any combination thereof; or
wherein the lung cancer is selected from the group consisting of non-small cell lung cancer such as lung adenocarcinoma, small cell lung cancer, and any combination thereof; or
wherein the breast cancer is selected from the group consisting of hormone receptor-positive breast cancer, triple-negative breast cancer, HER2-enriched breast cancer, and any combination thereof.

13. The method of claim 11, wherein the cancer is characterized by the presence of a KRAS mutation, optionally wherein the KRAS mutation is selected from the group consisting of G12A, G12C, G12D, G12E, G12F, G12I, G12R, G12S, G12V, G13A, G13C, G13D, G13E, G13R and any combination thereof.

14. The method of claim 1, wherein the cancer is a colorectal cancer, a lung cancer, a liver cancer, or a breast cancer, and the bacterium is Oscillibacter ruminantium or a recombinant Escherichia coli bacterium comprising a nucleic acid encoding cholesterol oxidase (CHOX).

15. The method of claim 1, wherein the 4-C-3 or bacterium is administered orally, or wherein the bacterium is administered via fecal microbiota transplant.

16. The method of claim 1, wherein the subject is a human, a non-human primate, a rodent, a canine, a feline, a bovine, or an equine.

17. A recombinant Escherichia coli TOP10 bacterium comprising a nucleic acid encoding cholesterol oxidase (CHOX), optionally, wherein the nucleic acid is operably linked to a promoter such that the bacterium expresses the CHOX under the control of the promoter.

18. The bacterium of claim 17, wherein the promoter is a constitutive promoter J23119, and wherein the CHOX is CHOX originating from Streptomyces species (such as Streptomyces fradiae, Streptomyces lavendulae, or Streptomyces cavourensis).

19. A composition comprising the bacterium of claim 17 and a pharmaceutically acceptable carrier.

20. The composition of claim 19, wherein the composition is in the form of a lyophilized powder, a freeze-dried preparation, a microencapsulation, an enteric-coated capsule, a tablet, a granule, a suspension, or any combination thereof.
